(19) United States
(12) Patent Application Publication
(10) Pub. No.: US 2025/0236901 A1
(43) Pub. Date: Jul. 24, 2025

(54) METHOD OF DETECTING A MICROORGANISM HAVING HIPPURICASE ACTIVITY

(71) Applicant: BIOSYNTH AG, Staad (CH)

(72) Inventors: URS SPITZ, St. Gallen (CH); CHUNYAN YAO, Radolfzell am Bodensee (DE)

(21) Appl. No.: 18/856,998

(22) PCT Filed: Apr. 21, 2023
(86) PCT No.: PCT/EP2023/060481

§ 371 Date: Oct. 15, 2024

(30) Foreign Application Priority Data
Apr. 22, 2022 (EP) 22169418.5

(51) Int. Cl. C12Q 1/34; C12Q 1/04

(52) U.S. Cl. CPC C12Q 1/34; C12Q 1/04; G01N 2333/205; G01N 2333/98

(57) ABSTRACT

The present invention relates to a method of detecting a microorganism having hippuricase activity comprising contacting a sample to be tested with p-hydroxyhippuric acid or a salt thereof, a detection reagent and an oxidizing reagent. The present invention further relates to a kit comprising p-hydroxyhippuric acid, a detection reagent and an oxidizing reagent, and further relates to the use of the kit for detecting a microorganism having hippuricase activity or for discriminating a microorganism having hippuricase activity from a microorganism without hippuricase activity or to the use of the kit in a method of detecting a microorganism having hippuricase activity.

### FIELD OF THE INVENTION

The present invention relates to a method of detecting a microorganism having hippuricase activity comprising contacting a sample to be tested with p-hydroxyhippuric acid or a salt thereof, a detection reagent and an oxidizing reagent. The present invention further relates to a kit comprising p-hydroxyhippuric acid, a detection reagent, and an oxidizing reagent, and further relates to the use of the kit for detecting a microorganism having hippuricase activity or for discriminating a microorganism having hippuricase activity from a microorganism without hippuricase activity or to the use of the kit in a method of detecting a microorganism having hippuricase activity.

### BACKGROUND OF THE INVENTION

Infections with microorganisms such as bacterial infections have a large impact on public health. Diseases can occur at any site caused by the microorganism itself or by the body's reaction to the presence of the microorganism. Usually, microorganisms are transferred to human bodies by air, food, water, contact with contaminated surfaces or living organisms.

For example, at a global scale, diarrheal diseases in humans are mainly caused by Campylobacter infections that can also result in gastroenteritis. Approximately 90% of human Campylobacter infection is caused by Campylobacter jejuni (C. jejuni) (WHO The Global View of Campylobacterioisis 2012; Center of Disease Control and Prevention 2019). Although often resulting in mild symptoms, Campylobacter infections can be fatal in young children, elderly, and immunosuppressed individuals. Infection is often foodborne, transmitted through contaminated meat, milk, water, or ice, that are not safely handled or properly heat-treated.

Campylobacter spp., e.g., C. jejuni and C. avium as well as other species such as Legionella spp., and Streptococcus agalactiae (known as group B Streptococci), Gardnerella vaginalis, Listeria monocytogenes, and Staphylococcus aureus produce the enzyme hippuricase. Hippuricase or the gene encoding hippuricase can be exploited for detection methods.

PCR-based assays focusing on the hipO gene have been developed. The assay has been applied to the analysis of environmental samples reaching a sensitivity of 0.01 pg/PCR, corresponding to 2-3 colony forming units (cfu) per ml. However, highly sensitive at the gene level, these assays do not provide information on the bacterial viability.

The hippurate hydrolysis test is a biochemical test which is based on the organism's ability to hydrolyze hippurate to benzoic acid and glycine by the action of the enzyme hippuricase. The reference assay for the detection of hippuricase-positive bacteria is a ninhydrin-based assay. The assay is based on the chromogenic reaction of ninhydrin with the amino group of glycine, one of the cleavage products of the hippuricase enzyme. The presence of hippuricase positive bacteria results in a blue coloration. However, the ninhydrin-based reaction is time-consuming and not specific to glycine because ninhydrin reacts with all molecules carrying alpha-amino groups that are present in the sample limiting its application to complex samples in nutrient-rich culture medium and resulting in false positives, i.e., the assay delivers a coloration indicating the presence of hippuricase-producing bacteria, although the bacteria are hippuricase negative.

Methods for detecting benzoic acid, the second product of hippurate hydrolysis, include a color reaction with ferric chloride (Ayers et al. J. Infect. Dis. 30: 388-389, 1922) and a gas-liquid chromatographic method (Wallace et al. J. Clin. Microbiol. 25: 1766-1768, 1987). The former method requires a long period of time and the latter method is highly equipment-dependent, requires trained personnel and specific sample preparation.

p-hydroxyhippuric acid is an N-acylglycine that is the 4-hydroxy derivate of hippuric acid, also known as N-(4-hydroxybenzoyl)glycine, 4-hydroxybenzoylglycine or 4-hydroxyhippuric acid (4-HHA). The enzymatic hydrolysis of 4-hydroxyhippuric acid to glycine and p-hydroxybenzoic acid has been involved in developing colorimetric determination of enzymes. Mostly, in the first step, 4-hydroxyhippuric acid was formed through the target enzyme. In the second step, 4-hydroxyhippuric acid was converted into p-hydroxybenzoic acid in presence of hippuricase. Furthermore, p-hydroxybenzoic acid was detected by formation of a quinoneimine dye through oxidative coupling with 4-aminoantipyrine catalyzed by peroxidase in presence of hydrogen peroxide or by sodium periodate. The absorbance of quinoneimine was measured to evaluate the activity of the target enzyme. For example, Kasahara et al. (Clin. Chem. 27 (11): 1922-1925, 1981) developed an assay of determining the activity of angiotensin-I converting enzyme and Saruta et al. (Clin. Chem. 32 (5): 748-751, 1986) developed a method of determining the activity of carboxypeptidase A. In these assays, purified hippuricase was used.

Edwards et al. (Analyst 732: 178, 1937) describe an assay for the detection of p-hydroxybenzoic acid by contacting the acid with Million's reagent (mercury dissolved in nitric acid and diluted with water). The presence of p-hydroxybenzoic acid was detected by the formation of a colored complex by reaction of the phenol group of p-hydroxybenzoic acid with Hg ions. However, this assay cannot be performed with living organisms, bears health risks for the personnel and is environmentally unacceptable.

Diaz et al. (Analytical Letters 41: 1-9, 2008) describe an assay for the detection of p-hydroxybenzoic acid based on inclusion of p-hydroxybenzoic acid into a cyclodextrin complex to enhance the fluorescent signal of p-hydroxybenzoic acid. Szopa et al. (Plant Cell Tiss. Organ. Cult. 113: 323-329 (2013) describe an assay for the detection of p-hydroxybenzoic acid based on HPLC from plant extracts. However, these techniques do not involve living organisms, requires specific sample preparation, trained personnel and are highly equipment dependent.

Despite the progresses mentioned above, there is still a need in the art for an assay for detecting hippuricase activity in living microorganisms that overcomes the limitations of the currently available assays.

### OBJECT OF THE INVENTION

In view of the above, it is therefore an object of the present invention to provide a method of detecting a microorganism having hippuricase activity that is not only sensitive and reliable but is also simple, sustainable, and fast.

### SUMMARY OF THE INVENTION

In one aspect, the invention provides a method of detecting a microorganism having hippuricase activity comprising:
a) providing a sample to be tested,
b) contacting and optionally incubating the sample from step a) with p-hydroxyhippuric acid or a salt thereof, a detection reagent, preferably a reagent for phenol detection, and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent, and
c) determining in the detection mixture of step b) the presence or absence of the detectable compound wherein the presence of the detectable compound indicates the presence of the microorganism having hippuricase activity.

In a further aspect, the invention provides a method of detecting a microorganism having hippuricase activity comprising:
a) providing a sample to be tested,
b) contacting and optionally incubating the sample from step a) with p-hydroxyhippuric acid or a salt thereof, a detection reagent, preferably a reagent for phenol detection, and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent, and
c) determining in the detection mixture of step b) the presence or absence of the detectable compound wherein the presence of the detectable compound indicates the presence of the microorganism having hippuricase activity
   wherein the oxidizing reagent comprises an oxidizing agent and a catalyst, wherein the oxidizing reagent preferably consists of an oxidizing agent and a catalyst.

In a further aspect, the invention provides a kit for detecting a microorganism with hippuricase activity comprising:
a) a first container comprising p-hydroxyhippuric acid, a second container comprising a detection reagent, and a third container comprising an oxidizing reagent, or
b) a container comprising p-hydroxyhippuric acid, a detection reagent, and an oxidizing reagent, or
c) a first container comprising p-hydroxyhippuric acid and a second container comprising a detection reagent and an oxidizing reagent, or
d) a first container comprising p-hydroxyhippuric acid and a detection reagent and a second container comprising an oxidizing reagent, and
e) optionally instructions for contacting a sample to be tested for comprising the microorganism with p-hydroxyhippuric acid, a detection reagent, and an oxidizing reagent.

In a further aspect, the invention provides a kit for detecting a microorganism with hippuricase activity comprising:
a) a first container comprising p-hydroxyhippuric acid, a second container comprising a detection reagent, a third container comprising a catalyst, and a fourth container comprising an oxidizing agent, or
b) a first container comprising p-hydroxyhippuric acid, a second container comprising a detection reagent and a catalyst, and a third container comprising an oxidizing agent, or
c) a first container comprising p-hydroxyhippuric acid and a second container comprising a detection reagent, a catalyst, and an oxidizing agent, or
d) a first container comprising p-hydroxyhippuric acid and a detection reagent, a second container comprising a catalyst, and a third container comprising an oxidizing agent, or
e) a first container comprising p-hydroxyhippuric acid and a detection reagent, and a second container comprising a catalyst, and an oxidizing agent, or
f) a first container comprising p-hydroxyhippuric acid, a second container comprising a detection reagent, and a third container comprising a catalyst and an oxidizing agent, or
(g) a first container comprising p-hydroxyhippuric acid, a detection reagent, and a catalyst, and a second container comprising an oxidizing agent, or
(h) a container comprising p-hydroxyhippuric acid, a detection reagent, a catalyst, and an oxidizing agent, and
i) optionally instructions for contacting a sample to be tested for comprising the microorganism with the p-hydroxyhippuric acid, the detection reagent, the catalyst, and the oxidizing agent.

In a further aspect, the invention relates to the use of the kits as described herein for detection of a microorganism having hippuricase activity, preferably hippuricase activity of an endogenous hippuricase enzyme, preferably in a sample.

In a further aspect, the invention relates to the use of the kits as described herein for discriminating a microorganism having hippuricase activity from a microorganism without hippuricase activity, preferably in a sample.

In a further aspect, the invention relates to the use of the kits as described herein in the method of detecting a microorganism having hippuricase activity as described herein.

### BRIEF DESCRIPTION OF THE FIGURES

FIG. 1 illustrates the reaction scheme for the detection of hippuricase activity in accordance with the present invention.

FIG. 2 shows absorbance signal development over time obtained with the method of the present invention for detecting bacteria having hippuricase activity. A: absorbance signal development of C. jejuni in comparison to C. coli, E. coli, and sterile control; final concentrations of 4-hydroxyhippuric acid (4-HHA) were 5 mM, 10 mM, and 20 mM. B: absorbance signal development of C. jejuni in comparison to C. coli, E. coli, and control without cells; final concentration of p-hydroxybenzoic acid was 1 mM.

FIG. 3 shows absorbance signal development over timer obtained by the methods of the present invention using samples with C. jejuni (C. j.) in comparison to C. coli (C. c.) at similar cell density. Cell suspensions in sterile 100 mM phosphate buffer pH 7.4 were incubated with 4-hydroxyhippuric acid for 1 h at 37° C., following the addition of 4-aminoantipyrine, horseradish peroxidase and followed with hydrogen peroxide. Absorbance at 505 nm was recorded for 15 minutes.

FIG. 4 compares absorbance signal development over time of A: C. jejuni and B: C. coli at similar cell densities. Cell suspensions in sterile 100 mM phosphate buffer pH 7.4 were incubated with 4-hydroxyhippuric acid for 1 h at 37° C. After incubation, 4-aminoantipyrine, peroxidase, glucose and glucose oxidase were added. Absorbance at 505 nm was recorded for 15 minutes.

FIG. 5A shows the absorbance intensity obtained by the methods of the present invention using samples with C. jejuni (C. j.) at 505 nm in comparison to C. coli (C. c.) at similar cell density. Endpoint absorbance was taken 10 minutes after all reagents were added. Cell suspensions in sterile 100 mM phosphate buffer pH 7.4 were incubated with 4-hydroxyhippuric acid for 1 h at 37° C., following the addition of 4-aminoantipyrine and NaIO₄. B shows the absorbance intensity of C. jejuni (C. j.) at 505 nm in comparison to C. coli (C. c.) at different cell densities as indicated over time. Cell suspensions in sterile 100 mM phosphate buffer pH 7.4 were incubated with 4-hydroxyhippuric acid for 1 h at 37° C., following the addition of 4-aminoantipyrine and NaIO₄.

FIG. 6 shows the linear correlation of absorbance intensity at 505 nm with optical density measured at 600 nm (OD₆₀₀) according to the methods of the present invention using samples with C. jejuni DSM 4688 and different oxidizing reagents. Cell suspensions in sterile 100 mM phosphate buffer pH 7.4 were incubated with 4-hydroxyhippuric acid for 1 h at 37° C., following the addition of 4-aminoantipyrine and different oxidizing reagents: A HRP/H₂O₂, B HRP/Glu/GOD, C NaIO₄. Endpoint absorbance after 10 min (absorbance of sterile control was subtracted) was evaluated.

FIG. 7 shows the range of the different oxidizing reagents added by detecting C. jejuni DSM 4688 (OD₆₀₀=0.4 to 0.5). Cell suspensions in sterile 100 mM phosphate buffer pH 7.4 were incubated with 4-hydroxyhippuric acid for 70 min. at 37° C., followed by the addition of 4-aminoantipyrine and various oxidizing reagents: A HRP/H₂O₂, B HRP/Glu/GOD, C NaIO₄. (w/o: without)

FIG. 8 shows the absorbance intensities obtained by the present methods using samples with Streptococcus agalactiae ATCC®12386 and Streptococcus pyogenes ATCC®19615. Cell suspensions in sterile 100 mM phosphate buffer pH 7.4 were incubated with 4-hydroxyhippuric acid for 80 min at 37° C., followed by the addition of 4-aminoantipyrine and various oxidizing reagents: A HRP/H₂O₂, B HRP/Glu/GOD, C NaIO₄. Endpoint absorbance after 15 min. (w.: with; w/o: without)

FIG. 9: shows absorbance signal development over time obtained with the method of the present invention for detecting bacteria having hippuricase and catalase activity or bacteria having hippuricase activity but no catalase activity. Absorbance signal development chromogenic reaction of p-hydroxybenzoic acid with 4-aminoantipyrine, horseradish peroxidase and H₂O₂ in the presence of bacterial cells with or without catalase, final concentration of p-hydroxybenzoic acid was 1 mM.

### DETAILED DESCRIPTION OF THE INVENTION

The present invention provides a method of detecting a microorganism having hippuricase activity which is based on the detection of a hippuricase cleavage product p-hydroxybenzoic acid.

The inventors specifically determined hippuricase activity of Campylobacter jejuni by contacting the microorganism with p-hydroxyhippuric acid, a detection reagent and an oxidizing reagent thereby producing a p-hydroxybenzoic acid based quinoneimine dye which could be determined by visual inspection and appeared very quickly within about a few minutes. The inventors also observed that this method was highly sensitive as a few bacterial colonies, e.g., an equivalent of OD₆₀₀ of about 0.1, and a small volume were sufficient to provide an intense signal. The reaction was unexpectedly fast thereby avoiding depletion of reaction reagents by other enzymes present in the microorganism, e.g., complete depletion of hydrogen peroxide by catalase. Thus, the present invention provides for a fast, yet specific, safe, and sustainable (lack of toxic reagents and organic solvents) method for detecting hippuricase-positive microorganisms such as C. jejuni with a chromogenic reaction delivering an easy-to-read visual positive result.

The invention provides a method of detecting a microorganism having hippuricase activity comprising:
a) providing a sample to be tested,
b) contacting and optionally incubating the sample from step a) with p-hydroxyhippuric acid or a salt thereof, a detection reagent, preferably a reagent for phenol detection, and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent, and
c) determining in the detection mixture of step b) the presence or absence of the detectable compound wherein the presence of the detectable compound indicates the presence of the microorganism having hippuricase activity.

Unless defined otherwise, all technical and scientific terms used herein have the same meaning as commonly understood by those of ordinary skill in the art to which this invention pertains.

The term “comprising” or “comprises” as used herein means “including, but not limited to”. The term is intended to be open-ended, to specify the presence of any stated features, elements, integers, steps, or components, but not to preclude the presence of addition of one or more other features, elements, integers, steps, components, or groups thereof. The term “comprising” or “comprises” thus includes the more restrictive terms “consisting of” and “consisting essentially of”. In one embodiment, the term “comprising” or comprises” as used throughout the application and in particular within the claims may be replaced by the term “consisting of”.

Terms which are indicated in singular herein relate to the term in plural as well unless it is explicitly stated otherwise herein and vice versa. For example, the term “kit” in singular as used herein also relates to the term “kits” in plural unless it is stated otherwise.

“p-hydroxyhippuric acid” as used herein refers to the following formula:

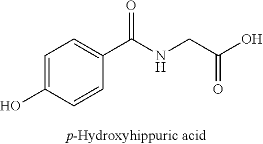 [SMILES: O=C(O)CNC(=O)c1ccc(O)cc1]

The term “p-hydroxyhippuric acid” as used herein is identical with the term “benzoyl glycocoll”, “benzoyl amidoacetic acid”, “benzamidoacetic acid”, “benzoylglycine”, “hippuric acid”, “4-hydroxyhippuric acid”, “4-HAA”, “4-Hydroxy-bz-gly-oh”, “2-[(4-hydroxyphenyl)formamido]acetic acid”, “2-(4-Hydroxybenzamido)acetic acid”, “4-Hydroxybenzoylglycine”, “N-(4-Hydroxybenzoyl)glycine”, “2-[(4-hydroxybenzoyl)amino]acetic acid”, “Glycine, N-(4-hydroxybenzoyl)-”, [(4-hydroxybenzoyl)amino]acetic acid”, “4-hydroxyhippurate”, “4-OH-hippuric acid”, “Bzo(4-OH)-Gly-OH”, “(4-hydroxybenzoyl)glycine”, “2-[(4-hydroxyphenyl)carbonylamino]acetic acid” or “hippurate”. These terms might be used interchangeable herein. The term p-hydroxyhippuric acid also includes salts and stereoisomers, particularly enantiomers of p-hydroxyhippuric acid.

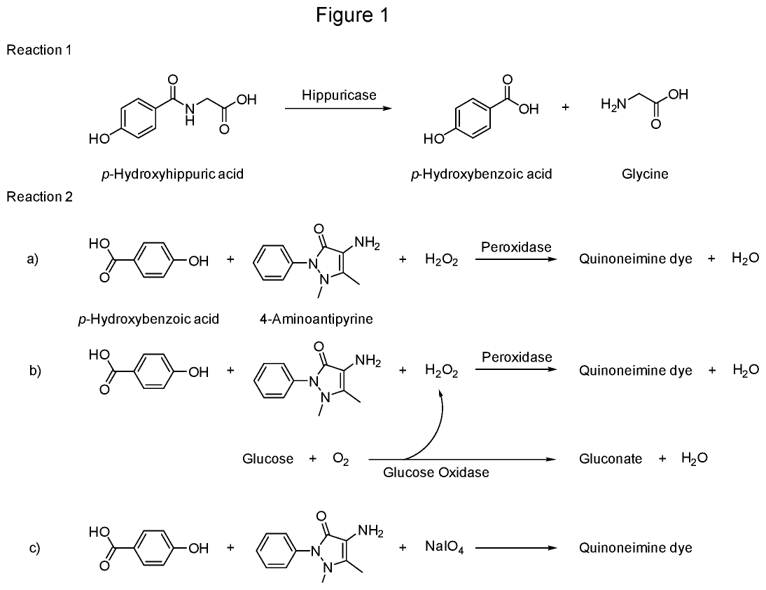

The term “hippuricase activity” refers to an enzymatic activity that results in the formation of p-hydroxybenzoic acid. For example, the “hippuricase activity” might refer to an enzymatic activity which comprises the enzymatic activity of the enzyme hippuricase (EC 3.5.1.32; also called “benzoylglycine amidohydrolase” or “hippurate hydrolase”). The “hippuricase activity” might also refer to an enzymatic activity which is identical to the enzymatic activity of the enzyme hippuricase (EC 3.5.1.32). The “hippuricase activity” might also refer to an enzymatic activity which comprises the enzymatic activity as shown in reaction 1 of FIG. 1, i.e., the formation of p-hydroxybenzoic acid and glycine. Preferably, the hippuricase activity is the activity of an endogenous enzyme, i.e., of an enzyme that originates from the microorganism by nature in contrast to, e.g., a recombinantly expressed enzyme. More preferably, the hippuricase activity is the activity of an endogenous enzyme that originates from an in vivo microorganism.

A “microorganism” as used herein refers to a microorganism of microscopic size. The microorganism has preferably an own metabolism. The microorganism might exist as a single cell or as a colony of cells. A microorganism might by an archaea, bacteria or eukaryote such as a protist, e.g., a slime mold, a fungus or a plant, e.g., an alga.

A “sample” or a “sample to be tested” as used herein refers to any kind of sample. The sample might be solid, or liquid, e.g., a suspension. The sample might also be gaseous, e.g., an aerosol. It might be derived from any source, such as from environment, from an industrial facility, from food, from water, from a dairy, from a commercial product, e.g., a commercial product intended for human or animal consumption, from a clinical facility, from air, e.g., indoor air, or from a subject, e.g., an animal such as a human. The sample derived from a subject might be from a body fluid such as blood, serum, bone marrow, salvia, vaginal fluid, urine, cerebrospinal fluid, placental or umbilical cord blood, lymphoid fluid, feces, or of a tissue.

A “detection reagent” as used herein refers to any reagent that shows the presence of p-hydroxybenzoic acid. A detection reagent might be a reagent for phenol detection, e.g., a reagent which modifies p-hydroxybenzoic acid to produce a detectable compound, preferably a compound which reacts via oxidative coupling to produce a chromogenic detectable compound or a compound which forms a chromogenic detectable compound by oxidative coupling. For example, the detection reagent reacts with p-hydroxybenzoic acid, preferably in the presence of an oxidizing reagent, and produces a detectable compound (e.g., a chromogenic, a fluorescent or a chemiluminescent detectable compound, preferably a chromogenic detectable compound). In one embodiment, the detection reagent is not a metal or metal ion, e.g., Fe, e.g., Fe(II) or Hg, e.g., Hg(I).

In principle, the term “determining the presence or absence of the detectable compound”, “detection” or “detecting” as used herein refers to any method of detection which is specific for the detectable compound. The skilled person will understand that the exact method may depend on the detection reagent that is used to produce the detectable compound. Determining the presence or absence of the detectable compound might be qualitatively such as by visual inspection of a signal such as a color and/or quantitatively. Preferably, determining the presence or absence of the detectable compound comprises detecting a specific absorbance of a detectable compound. Alternatively, determining the presence or absence of the detectable compound comprises measuring light emitted by a detectable compound, e.g., by chemiluminescence or fluorescence. In yet a further alternative embodiment, determining the presence or absence of the detectable compound comprises detection of the detectable compound by odor, precipitation, phase-separation, heat, light or sound.

An “oxidizing reagent”, “oxidant” or “oxidizer” as used herein refers to a substance, compound or molecule that has the ability to oxidize other substances, compound or molecule, i.e., it accepts the electrons of other substances compounds or molecules. Common oxidizing reagents include but are not limited to oxygen, hydrogen peroxide or halogens which are particularly useful for the purpose of the present invention. In one embodiment, the oxidizing reagent is not a metal or metal ion, e.g., Fe, e.g., Fe(II) or Hg, e.g., Hg(I) or air oxygen.

“Oxidative coupling” as used herein refers to a chemical reaction of two molecular groups of a substance, compound, or molecule through an oxidative process to produce a covalent chemical bond, particularly a covalent chemical bond between carbons or preferably a covalent chemical bond between a carbon and a nitrogen. The covalent chemical bond may be a single, double or triple bond, preferably an imino bond. In a preferred embodiment, oxidative coupling refers to the extension of conjugated bonds, i.e., alternated single and double bonds, of p-hydroxybenzoic acid.

In one embodiment, the method of the invention does not comprise the addition or presence of an organic solvent such as, e.g., o-phenanthroline.

In one embodiment, the sample of step a) comprises a microorganism.

The method of the invention relates in step b) to contacting and optionally incubating the sample from step a) with p-hydroxyhippuric acid or a salt thereof, a detection reagent and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent.

In one embodiment, p-hydroxyhippuric acid or a salt thereof in step b) is present at a final concentration suitable for carrying out the method including but not limited to a final concentration of about 1 mM to 25 mM, or about 3 mM to 20 mM, or about 5 mM to 15 mM or about 7 mM to 10 mM. In one embodiment, p-hydroxyhippuric acid or a salt thereof in step b) is present at a final concentration of about 10 mM.

In one embodiment, the method of the invention relates in step b) to contacting and, optionally incubating, the sample with the p-hydroxyhippuric acid or the salt thereof followed by adding, preferably adding simultaneously, the detection reagent and the oxidizing reagent.

In another embodiment, the method of the invention relates in step b) to contacting and, optionally incubating, the sample with the p-hydroxyhippuric acid or the salt thereof and the detection reagent, preferably simultaneously, followed by adding the oxidizing reagent.

In one aspect of the method of the invention, the oxidizing reagent comprises a halogen, e.g., a halogen salt, e.g., a perchlorate salt, perbromate salt or periodate salt preferably a periodate salt, more preferably sodium periodate.

In one embodiment, the oxidizing reagent is sodium periodate (NaIO₄). In one embodiment, the oxidizing agent, preferably sodium periodate, is present in step b) at a final concentration suitable for carrying out the method including but not limited to a final concentration of about 0.5 mM to 10 mM, or about 1 mM to 8 mM, or about 2 mM to about 4 mM. In one embodiment, the oxidizing agent, preferably sodium periodate, in step b) is present at a final concentration of about 2 mM. In one embodiment, the oxidizing agent, preferably sodium periodate, in step b) is present at a final concentration of about 4 mM. In one embodiment, the oxidizing agent, preferably sodium periodate, in step b) is present at a final concentration of about 8 mM.

In another aspect, of the method of the invention, the oxidizing reagent comprises an oxidizing agent and a catalyst. In a preferred embodiment, the oxidizing agent is hydrogen peroxide and/or a hydrogen peroxide-generating system. Preferably, the hydrogen peroxide-generating system is an oxidase and a substrate, wherein more preferably the oxidase is a glucose oxidase (GOD), and the substrate is glucose.

In one embodiment, the oxidizing agent is hydrogen peroxide. In one embodiment, the oxidizing agent, preferably hydrogen peroxide, is present in step b) at a final concentration suitable for carrying out the method including but not limited to a final concentration of about 0.5 mM to 20 mM, or about 1 mM to about 16 mM, or about 1 mM to 8 mM, or about 1 mM to 4 mM. In one embodiment, the oxidizing agent, preferably hydrogen peroxide, is present in step b) at a final concentration of about 1 mM. In one embodiment, the oxidizing agent, preferably hydrogen peroxide, is present in step b) at a final concentration of about 2 mM. In one embodiment, the oxidizing agent, preferably hydrogen peroxide, is present in step b) at a final concentration of about 4 mM.

In one embodiment, the oxidizing agent is a hydrogen peroxide-generating system, preferably comprising an oxidase preferably glucose oxidase. In one embodiment, the oxidase is present in step b) at a final concentration suitable for carrying out the method including but not limited to a final concentration of about 0.25 u/mL to 10 U/mL, or about 0.5 U/mL to 5 U/mL, or about 1 U/mL to 4.5 U/mL, or about 2 U/mL to about 4 U/mL. In one embodiment, the oxidase is present in step b) at a final concentration of about 1 U/mL. In one embodiment, the oxidase is present in step b) at a final concentration of about 2 U/mL. In one embodiment, the oxidase is present in step b) at a final concentration of about 4 U/mL. In one embodiment, glucose in step b) is present at a final concentration suitable for carrying out the method, preferably a final concentration of about 1 mM to 20 mM, or about 5 mM to about 15 mM, or about 7 mM to 10 mM. In one embodiment, glucose in step b) is present at a final concentration about 10 mM.

In one embodiment, the catalyst is a horseradish peroxidase. In one embodiment, the catalyst, preferably horseradish peroxidase, is present in step b) at a final concentration suitable for carrying out the method including but not limited to a final concentration of about 0.5 u/mL to 10 U/mL, or about 1 U/mL to 5 U/mL, or about 2 U/mL to 4.5 U/mL. In one embodiment, the catalyst, preferably horseradish peroxidase, is present in step b) at a final concentration of about 4.5 U/mL.

Specific embodiments of these different aspects of the method of the invention as well as kits comprising the oxidizing reagent or the oxidizing agent and the catalyst and uses of these kits in the method of the invention as well as further uses of the kit are disclosed herein in detail further below. The following embodiments of the method of the invention apply to the different aspects of the method of the invention as disclosed herein.

In one embodiment of the method of the invention, the detection reagent refers to any reagent that can be used to detect p-hydroxybenzoic acid. Such detection reagents are known to the skilled person. For example, a detection reagent might be a reagent for phenol detection, e.g., a compound which modifies, e.g., reacts with, e.g., via oxidative coupling, p-hydroxybenzoic acid to produce a chromogenic detectable compound. The detection reagent in this regard might be selected from the group consisting of 4-aminoantipyrine or a salt thereof, 3-methyl-2-benzothiazolinone hydrazone or a salt thereof and N-methyl-N-phenyl-3-sulfophenylenediamine or a salt thereof. A preferred detection reagent is selected from the group consisting of 4-aminoantipyrine or a salt thereof and 3-methyl-2-benzothiazolinone hydrazone or a salt thereof. A particularly preferred detection reagent is 4-aminoantipyrine. Alternatively, a detection reagent might be a compound which modifies, e.g., reacts with, p-hydroxybenzoic acid to produce a chemiluminescent detectable compound. Alternatively, a detection reagent might be a compound, e.g., a label, which modifies, e.g., reacts with p-hydroxybenzoic acid to produce a fluorescent detectable compound.

In one embodiment, the detection reagent in step b) is present at a final concentration suitable for carrying out the method including but not limited to a final concentration of about 0.1 mM to 5 mM, or about 0.2 mM to 3 mM, or about 0.5 mM to 2 mM. In one embodiment, the detection reagent in step b) is present at a final concentration of about 0.5 mM or about 2 mM.

In one embodiment, the oxidizing reagent is not air oxygen.

In a further embodiment of the method of the invention, step b) and step c) are direct consecutive steps. In other words, step c) is performed without further steps in between steps b) and c).

In one embodiment of the method of the invention, in step c) determining in the detection mixture of step b) the presence or absence of the detectable compound comprises any method suitable for detecting the detectable compound. The skilled person knowns that a specific method depends on the specific chemical and physical properties of the detection reagent and/or the detectable compound. For example, determining the presence or absence of the detectable compound may comprise fluorescence, absorbance, chemiluminescence, odor, precipitation, phase-separation, heat, light or sound, preferably absorbance. In one embodiment, the detection method does not comprise detection of a metal complex, such as Fe or Hg complex, i.e., the detectable compound is not a metal complex, e.g., a Fe or Hg complex. For example, determining the presence or absence of the detectable compound in in the detection mixture comprises comparison to a reference or control not comprising the detectable compound, e.g., a control not comprising the microorganism, i.e., a sterile control, or a control comprising a microorganism without hippuricase activity such as Streptococcus pyogenes, Campylobacter coli or Escherichia coli, preferably Streptococcus pyogenes or Campylobacter coli. In one embodiment, the signal of the detectable compound in the detection mixture is different from the signal of the control or reference. In another embodiment, the detectable compound in the detection mixture shows a signal that is absent in the control or reference.

In a preferred embodiment of the method of the invention, determining the presence or absence of the detectable compound in step c) comprises detecting absorbance, preferably absorbance of a chromogenic detectable compound, more preferably of a chromogenic detectable compound which is produced in the presence of the oxidizing reagent, preferably by oxidative coupling of p-hydroxybenzoic acid with the detection reagent. An example of a chromogenic detectable compound is a quinone or a derivative thereof, e.g., a quinoneimine.

In a specific embodiment of the method of the invention, the absorbance of the detection mixture is compared to a reference or control not comprising the detectable compound, e.g., a control not comprising the microorganism, i.e., a sterile control, or a control comprising a microorganism without hippuricase activity such as Streptococcus pyogenes, Campylobacter coli or Escherichia coli, preferably Streptococcus pyogenes or Campylobacter coli. In one embodiment, the detectable compound might show absorbance that is different or absent from the absorbance of the control or reference. The absorbance can be determined by any method known in the art and may be qualitatively or quantitatively. For example, the absorbance might be determined by visual inspection or by measuring. In a preferred embodiment, the absorbance of the detectable compound is measured at wavelength in a range of about 500 nm to about 600 nm, preferably about 505 nm to about 515 nm. Methods for measuring the absorbance are generally known to the skilled person and may comprise measuring the absorbance with a spectrometer or photometer, e.g., a UV/VIS spectrophotometer.

In a particular preferred embodiment of the method of the invention, the detection reagent is 4-aminoantipyrine or a salt thereof and the absorbance of the detectable compound is measured in a range of about 500 nm to about 515 nm, preferably about 505 nm.

In a preferred embodiment, the detection reagent is 3-methyl-2-benzothiazolinone hydrazone or a salt thereof and the absorbance of the detectable compound is measured in a range of about 500 nm to about 515 nm, preferably about 505 nm (Rodríguez-López et al. Anal Biochem. 1994 January; 216(1):205-12).

In a further embodiment of the method of the invention, the microorganism is a bacterium. In a preferred embodiment, the bacterium comprises a catalase, i.e., the bacterium is catalase positive. In one embodiment “providing a sample to be tested” as used herein refers to a taken sample, e.g., a sample taken from a subject, food or environment as described herein. In a further preferred embodiment, the microorganism is a bacterium preferably from Campylobacter species, Gardnerella species, Streptococcus species, Staphylococcus species, Legionella species and/or Listeria species. In yet a further preferred embodiment, the microorganism is selected from the group consisting of Campylobacter jejuni, Campylobacter avium, Gardnerella vaginalis, Streptococcus group B (Streptococcus agalactiae), preferably bovine Streptococcus group B, Staphylococcus aureus, Streptococcus agalactiae and Listeria monocytogenes. In a particular preferred embodiment, the microorganism is from Campylobacter species, preferably Campylobacter jejuni. In another preferred embodiment, the microorganism is from Streptococcus species, preferably Streptococcus agalactiae.

In a further preferred embodiment of the method of the invention, the sample in step a) is from a subject such as an animal. The animal may be an animal that lives in close relationship with a human such a farm animal or a pet. Examples of animals are dogs, cats, birds, cattle, sheep swine, goat, or poultry. In a preferred embodiment, the subject is a human. The sample of the subject may be derived from any fluid, such as blood, serum, bone marrow, salvia, vaginal fluid, urine, cerebrospinal fluid, placental or umbilical cord blood, lymphoid fluid, feces, and/or from a tissue of the subject.

In one embodiment of the method of the invention, the sample in step a) is from a subject as described herein and the method further comprises a step d) of administering a medicament to the subject if the presence of a detectable compound has been determined in step c). The skilled person knows which medicament will be effective for the microorganism detected by the method of the invention. For example, a medicament may be an electrolyte solution and/or an antibiotic such as azithromycin or ciprofloxacin.

In a further preferred embodiment of the method of the invention, the sample in step a) is from environment. For examples the sample is from water, e.g., drinking water; dung; stables such as stables from farm animals; dust and/or industrial facilities such as floor, devices, facilities; sand or other sediments; soil or industrial waste.

In a further preferred embodiment of the method of the invention, the sample in step a) is from a product intended for human or animal consumption such as food. Examples are meat, e.g., from poultry, cattle, sheep, swine and/or goat; dairy product, e.g., milk (raw or pasteurized); vegetables; fish, e.g., shellfish; or fruits.

In one embodiment, the sample in step a) comprises a microorganism.

In one embodiment of the method of the invention, the sample to be tested in step a) is contacted in step b) without further pre-treatment step. In a specific embodiment, the sample is a sample from a subject.

In an alternative embodiment of the method of the invention, step a) comprises one or more further pre-treatment step(s). For example, step a) comprises a culturing step, optionally wherein a pre-enrichment step precedes the culturing step. In a specific embodiment, the sample is from environment or from a product intended for human or animal consumption. The skilled person knows that a potential pre-treatment step such as a culturing step and/or pre-enrichment step may depend on the microorganism to be detected and/or the source of the sample and/or the degree of contamination of the source or the sample (e.g., FDA, Bacteriological Analytical Manual (BAM), 8^{th} edition, Revision A, 1998, Chapter 7: https://www.fda.gov/food/laboratory-methods-food/bam-chapter-7-campylobacter). For example, samples from subjects may not need a pre-enrichment step and may be directly cultured while samples from food or environment may need a pre-enrichment step, e.g., to facilitate recovery of damaged cells.

The culturing step may comprise aerobic or microaerobic conditions, preferably microaerobic conditions. “Microaerobically”, “microaerobic conditions” or “under microaerobic atmosphere” are used herein interchangeably and refer to an oxygen level of in a range of about 2% to about 10%, for example about 5% to about 7%. Preferably, the culturing step may comprise culturing the sample in a suitable culture medium. Suitable culture media are known to the skilled person, e.g., Bolton Broth (e.g., Oxoid AM7526 or Malthus Diagnostics LAB-135, Malthus Diagnostics, North Ridgeville, OH 216-327-2585). The culture medium may further comprise supplements such as lysed horse blood, e.g., 5% lysed horse blood, and/or an antibiotic. In a preferred embodiment, the culturing step comprises microaerobic conditions at a temperature in a range of about 40° C. to about 45° C., more preferably about 41° C., about 42° C., about 43° C. or about 44° C., most preferably at about 42° C., for about 20 hours to about 48 hours, preferably about 24 hours to about 44 hours. The culturing step might further include a step of isolating the microorganism comprising striking the cultured sample on a selective agar plate specific for the growth of the microorganism to be detected, e.g., blood agar such as Columbia agar, and incubating the agar plate at temperature of about 37° C. to 42° C., preferably for about 24 to 48 hours. The above culturing conditions are particularly useful for detecting microorganisms such as Campylobacter spp. Specific examples of selective agars for Campylobacter spp. include but are not limited to Columbia agar, modified charcoal cefoperazone deoxycholate (mCCD agar), Abeyta-Hunt-Bark (AHB) agar, Preston agar, Skirrow agar or Butzler agar.

Alternatively, the culturing step comprises culturing the sample under aerobic conditions, e.g., air, preferably, under about 21% oxygen and about 0.05% carbon dioxide, for about 40 hours to about 48 hours, e.g., for about 45 hours, at a temperature of about 35° C. to about 45° C., preferably about 37° C., about 42° C., about 43° C. or about 44° C., more preferably at about 41.5° C.

Alternatively, the culturing step comprises culturing the sample with about 20% oxygen and about 5% carbon dioxide, for about 40 hours to about 48 hours, e.g., for about 45 hours, at a temperature of about 35° C. to about 45° C., preferably about 37° C., about 42° C., about 43° C. or about 44° C., more preferably at about 41.5° C.

In yet a further embodiment of the method of the invention, a pre-enrichment step precedes the culturing step. The skilled person knowns that the pre-enrichment step may vary depending on the specific sample and may comprise, e.g., filtering, culturing and/or homogenization.

In a specific embodiment of the method of the invention, pre-treatment of the sample comprises culturing the sample under microaerobic conditions for about 3 to about 6 hours, preferably about 4 hours at a temperature of about 30° C. to about 38° C., preferably about 33° C., about 34° C., about 35° C., about 36° C. or about 37° C. In one embodiment, pre-enrichment may comprise culturing the sample in a suitable culture medium microaerobically for about 3 hours to about 4 hours, preferably about 3 hours at a temperature of about 28° C. to about 35° C., preferably about 30° C., about 31° C., or about 32° C., preferably followed by a culturing step comprising culturing the sample in a suitable culture medium microaerobically for about 1 to about 3 hours, preferably about 2 hours at a temperature of about 35° C. to about 38° C., preferably about 36° C. or about 37° C. A suitable culture medium is known to the skilled person, e.g., Bolton Broth (e.g., Oxoid AM7526 or Malthus Diagnostics LAB-135, Malthus Diagnostics, North Ridgeville, OH 216-327-2585). The above pre-treatment conditions are particularly useful for detecting microorganisms such as Campylobacter spp.

In one embodiment of the method of the invention, the microorganism is a bacterium, preferably Campylobacter spp., and preferably the sample is from a food or environment as described herein, and pre-enrichment comprises culturing the sample under microaerobic atmosphere for about 3 to about 6 hours, preferably about 4 hours at a temperature of about 35° C. to about 38° C., preferably about 37° C. followed by a culturing step comprising culturing the sample microaerobically at a temperature in a range of about 40° C. to about 42° C., more preferably about 41.5° C. for about 40 hours to about 48 hours, preferably about 44 hours including isolation of the microorganism with a selective agar specific for the microorganism to be detected, e.g. blood agar such as Columbia agar. In a further preferred embodiment, the pre-enrichment and culturing step is performed as described in ISO 10272-1:2017 and/or ISO 11133:2014.

In another embodiment of the method of the invention, the microorganism is a bacterium, preferably Campylobacter spp., and preferably the sample is from environment as described herein, more preferably from water. Preferably, a pre-enrichment step such as filtering is performed, preferably with a positively charged filter, e.g., Zetapor®, prior to subjecting the filter to a culturing step as described herein. In a further preferred embodiment pre-enrichment and culturing is performed as described in ISO 17995:2019.

As stated above, in one aspect, the method of the invention relates in step b) to contacting and optionally incubating, preferably at a temperature in a range of about 36° C. to about 38° C., preferably at about 37° C., for about 1 hour or about 1 hour and 10 minutes, the sample from step a) with p-hydroxyhippuric acid or a salt thereof, and a detection reagent and an oxidizing reagent to obtain a detection mixture.

The embodiments of the inventive method disclosed herein for step a) including the one or more pre-treatment step(s) and/or for c) also apply to this aspect of the invention.

In a preferred embodiment, the oxidizing reagent is a halogen, preferably a halogen salt, preferably a periodate salt, more preferably sodium periodate. In one embodiment, the detection reagent is a reagent for phenol detection as described herein, e.g., selected from the group consisting of 4-aminoantipyrine or a salt thereof, 3-methyl-2-benzothiazolinone hydrazone or a salt thereof, and N-methyl-N-phenyl-3-sulfophenylenediamine or a salt thereof. In a preferred embodiment, the detection reagent is selected from the group consisting of 4-aminoantipyrine or a salt thereof and 3-methyl-2-benzothiazolinone hydrazone or a salt thereof. In a particularly preferred embodiment, the detection reagent is 4-aminoantipyrine or a salt thereof.

In one embodiment, p-hydroxyhippuric acid or a salt thereof in step b) is present at a final concentration suitable for carrying out the method including but not limited to a final concentration of about 1 mM to 25 mM, or about 3 mM to 20 mM, or about 5 mM to 15 mM or about 7 mM to 10 mM. In one embodiment, p-hydroxyhippuric acid or a salt thereof in step b) is present at a final concentration of about 10 mM.

In one embodiment, the oxidizing reagent is sodium periodate (NaIO₄). In one embodiment, the oxidizing reagent, preferably sodium periodate, is present in step b) at a final concentration suitable for carrying out the method including but not limited to a final concentration of about 0.5 mM to 10 mM, or about 1 mM to 8 mM, or about 2 mM to about 4 mM. In one embodiment, the oxidizing agent, preferably sodium periodate, in step b) is present at a final concentration of about 2 mM. In one embodiment, the oxidizing agent, preferably sodium periodate, in step b) is present at a final concentration of about 4 mM. In one embodiment, the oxidizing agent, preferably sodium periodate, in step b) is present at a final concentration of about 8 mM.

In one embodiment, the detection reagent in step b) is present at a final concentration suitable for carrying out the method including but not limited to a final concentration of about 0.1 mM to 5 mM, or about 0.2 mM to 3 mM, or about 0.5 mM to 2 mM. In one embodiment, the detection reagent in step b) is present at a final concentration of about 0.5 mM or about 2 mM.

In one embodiment, the oxidizing reagent is not air oxygen.

In this aspect of the method, step b) may comprise contacting the sample of step a) with p-hydroxyhippuric acid or the salt thereof and the detection reagent and the oxidizing reagent simultaneously and optionally incubating the detection mixture.

In a further embodiment, step b) may comprise contacting the sample of step a) with p-hydroxyhippuric acid or the salt thereof and the detection reagent and the oxidizing reagent each separately and optionally incubating the detection mixture.

In a further embodiment, step b) may comprise contacting the sample of step a) with p-hydroxyhippuric acid or the salt thereof; optionally incubating followed by adding the detection reagent and the oxidizing reagent. The detection reagent and the oxidizing reagent may be added simultaneously or separately.

The invention further relates to a kit comprising p-hydroxyhippuric acid or a salt thereof, a detection reagent, and/or an oxidizing reagent, e.g., for contacting a sample to be tested for the presence or absence of a microorganism having hippuricase activity.

In one embodiment, the detection reagent is a reagent for phenol detection as described herein, e.g., selected from the group consisting of 4-aminoantipyrine or a salt thereof, 3-methyl-2-benzothiazolinone hydrazone or a salt thereof, and N-methyl-N-phenyl-3-sulfophenylenediamine or a salt thereof. In a preferred embodiment, the detection reagent is selected from the group consisting of 4-aminoantipyrine or a salt thereof and 3-methyl-2-benzothiazolinone hydrazone or a salt thereof. In a particularly preferred embodiment, the detection reagent is 4-aminoantipyrine or a salt thereof.

In yet a further preferred embodiment, the oxidizing reagent is a halogen, such as a halogen salt, preferably a periodate salt, preferably sodium periodate.

In a further embodiment, the kit further comprises one or more auxiliary agents such as a preservative, an antioxidant, water, a stabilizer or a buffer.

In one embodiment of the kit of the invention, one or more of the reagents of the kit such as p-hydroxyhippuric acid or a salt thereof, the detection reagent, the oxidizing reagent and/or the auxiliary agent is/are present in liquid form, e.g., as solution, suspension, emulsion or in oily or aqueous vehicles.

In a preferred embodiment of the kit of the invention, one or more of the reagents of the kit such a p-hydroxyhippuric acid or a salt thereof, the detection reagent, the oxidizing reagent and/or the auxiliary agent is/are present in solid form, e.g., as a powder, granules or a tablet, and the kit optionally comprises one or more further container(s) comprising water or a buffer such as a phosphate buffer for reconstitution. In a more preferred embodiment, the p-hydroxyhippuric acid is present in solid form.

In one embodiment of the invention, the kit comprises a first container comprising p-hydroxyhippuric acid, a second container comprising the detection reagent, and a third container comprising the oxidizing reagent, and optionally instructions for contacting a sample to be tested for comprising the microorganism with the p-hydroxyhippuric acid, the detection reagent, and the oxidizing reagent.

In another embodiment, the kit comprises a container comprising p-hydroxyhippuric acid, the detection reagent, and the oxidizing reagent, and optionally instructions for contacting a sample to be tested for comprising the microorganism with the p-hydroxyhippuric acid, the detection reagent, and the oxidizing reagent.

In another embodiment, the kit comprises a first container comprising p-hydroxyhippuric acid and a second container comprising the detection reagent and the oxidizing reagent, and optionally instructions for contacting a sample to be tested for comprising the microorganism with the p-hydroxyhippuric acid, the detection reagent, and the oxidizing reagent.

In yet another embodiment, the kit comprises a first container comprising p-hydroxyhippuric acid and the detection reagent and a second container comprising the oxidizing reagent, and optionally instructions for contacting a sample to be tested for comprising the microorganism with the p-hydroxyhippuric acid, the detection reagent, and the oxidizing reagent.

In a further embodiment, the kit further comprises a container comprising a culture medium suitable for one or more pre-treatment steps, e.g., for culturing and/or pre-enrichment of the sample to be tested as described herein.

A “container” as used herein may be any container that is suitable for carrying the reagents. The skilled person will know suitable containers and will be aware that the type of container depends on the reagent and its physical form, e.g., liquid or solid. In a preferred embodiment of the kit of the invention, the container is a vial, ampoule, and/or a membrane such as a nitrocellulose membrane. The membrane may be in the form of a strip or a disk. The membrane may comprise one or more of the reagents of the kit as described herein. For example, the membrane may comprise, e.g., may be saturated, with p-hydroxyhippuric acid or a salt thereof. Alternatively, the membrane may comprise, e.g., may be saturated, with p-hydroxyhippuric acid or a salt thereof and the detection reagent.

In a further embodiment, the kit further comprises a further container suitable for incubating and/or culturing the sample, such as a tube or a vial.

The invention further relates to the use, preferably ex vivo, in vitro, of the kit for detection of a microorganism having hippuricase activity, preferably hippuricase activity of an endogenous hippuricase enzyme of the microorganism, preferably in a sample.

In one embodiment, the microorganism is a bacterium. In a preferred embodiment, the bacterium comprises a catalase, i.e., the bacterium is catalase positive. In a further preferred embodiment, the microorganism is a bacterium preferably from Campylobacter species, e.g., Campylobacter jejuni or Campylobacter avium; Gardnerella species, e.g. Gardnerella vaginalis, Streptococcus species, e.g., Streptococcus group B (Streptococcus agalactiae), preferably bovine Streptococcus group B; Staphylococcus species, e.g., Staphylococcus aureus; Legionella species and/or Listeria species, e.g., Listeria monocytogenes. In a more preferred embodiment, the microorganism is from Campylobacter species, preferably Campylobacter jejuni. In a more preferred embodiment, the microorganism is from Streptococcus species, preferably Streptococcus agalactiae.

In a further embodiment, the sample is from a subject such as an animal. The animal may be an animal that lives in close relationship with a human such a farm animal or a pet. Examples of animals are dogs, cats, birds, cattle, sheep swine, goat or poultry. In a preferred embodiment, the subject is a human. The sample of the subject may be from a body fluid, such as blood, serum, bone marrow, salvia, vaginal fluid, urine, cerebrospinal fluid, placental or umbilical cord blood, lymphoid fluid, feces, and/or from a tissue of the subject.

In a further preferred embodiment, the sample is from environment. For example, the sample might be from water such as drinking water; dung; stables such as stables from farm animals; dust, industrial facilities such as floor, devices, and facilities; sand or other sediments; soil or industrial waste.

In a further preferred embodiment, the sample is from a product intended for human consumption such as food. Examples are meat, e.g., from poultry, cattle, sheep, swine and/or goat; dairy product, e.g., milk (raw or pasteurized); vegetables; fish, e.g., shellfish; or fruits.

In one embodiment, the sample comprises a microorganism.

The invention further relates to the use, preferably ex vivo or in vitro, of the kit for discriminating a microorganism having hippuricase activity from a microorganism without hippuricase activity, preferably in a sample.

In one embodiment, the microorganism is a bacterium. In a preferred embodiment, the bacterium comprises a catalase, i.e., the bacterium is catalase-positive. In a further preferred embodiment, the microorganism is a bacterium preferably from Campylobacter species, e.g., Campylobacter jejuni or Campylobacter avium; Gardnerella species, e.g. Gardnerella vaginalis, Streptococcus species, e.g., Streptococcus group B (Streptococcus agalactiae), preferably bovine Streptococcus group B; Staphylococcus species, e.g., Staphylococcus aureus; Legionella species and/or Listeria species, e.g., Listeria monocytogenes. In a more preferred embodiment, the microorganism is from Campylobacter species, preferably Campylobacter jejuni. In a more preferred embodiment, the microorganism is from Streptococcus species, preferably Streptococcus agalactiae.

In a further embodiment, the sample is from a subject such as an animal. The animal may be an animal that lives in close relationship with a human such a farm animal or a pet. Examples of animals are dogs, cats, birds, cattle, sheep swine, goat or poultry. In a preferred embodiment, the subject is a human. The sample of the subject may be from a body fluid, such as blood, serum, bone marrow, salvia, vaginal fluid, urine, cerebrospinal fluid, placental or umbilical cord blood, lymphoid fluid, feces, and/or from a tissue of the subject.

In a further preferred embodiment, the sample is from environment. For example, the sample might be from water such as drinking water; dung; stables such as stables from farm animals; dust from industrial facilities such as floor, devices, and facilities; sand or other sediments; soil or industrial waste.

In a further preferred embodiment, the sample is from a product intended for human consumption such as food. Examples are meat, e.g., from poultry, cattle, sheep, swine and/or goat; dairy product, e.g., milk (raw or pasteurized); vegetables; fish, e.g., shellfish; or fruits.

In one embodiment, the sample comprises a microorganism.

The invention further relates to the use, preferably ex vivo or in vitro, of the kit of the invention in the method as described herein.

The invention further relates to a method for detection of a microorganism having hippuricase activity, preferably hippuricase activity of an endogenous hippuricase enzyme, comprising the use of the kit of the invention.

The invention further relates to a method for discriminating a microorganism having hippuricase activity from a microorganism without hippuricase activity, comprising the use of the kit of the invention.

The invention further relates to a method for detecting a microorganism having hippuricase activity, comprising the use of the kit of the invention.

As stated above, in another aspect, the method of the invention relates in step b) to contacting and optionally incubating, preferably at a temperature in a range of about 36° C. to about 38° C., preferably at about 37° C., for about 1 hour or about 1 hour and 10 minutes, the sample from step a) with p-hydroxyhippuric acid or a salt thereof, a detection reagent, preferably a reagent for phenol detection, and an oxidizing reagent to obtain a detection mixture wherein the oxidizing reagent comprises an oxidizing agent and a catalyst.

The embodiments of the inventive method disclosed herein for step a) including the one or more pre-treatment step(s) and/or for c) also apply to this aspect of the invention.

In a preferred embodiment, the oxidizing agent is hydrogen peroxide and/or a hydrogen peroxide-generating system wherein preferably the hydrogen peroxide-generating system is an oxidase and a substrate, wherein more preferably the oxidase is a glucose oxidase, and/or the substrate is glucose. In a further preferred embodiment, the catalyst is a peroxidase, preferably horseradish peroxidase.

In one embodiment, p-hydroxyhippuric acid or a salt thereof in step b) is present at a final concentration suitable for carrying out the method including but not limited to a final concentration of about 1 mM to 25 mM, or about 3 mM to 20 mM, or about 5 mM to 15 mM or about 7 mM to 10 mM. In one embodiment, p-hydroxyhippuric acid or a salt thereof in step b) is present at a final concentration of about 10 mM.

In one embodiment, the oxidizing agent is hydrogen peroxide. In one embodiment, the oxidizing agent, preferably hydrogen peroxide, is present in step b) at a final concentration suitable for carrying out the method including but not limited to a final concentration of about 0.5 mM to 20 mM, or about 1 mM to about 16 mM, or about 1 mM to 8 mM, or about 1 mM to 4 mM. In one embodiment, the oxidizing agent, preferably hydrogen peroxide, is present in step b) at a final concentration of about 1 mM. In one embodiment, the oxidizing agent, preferably hydrogen peroxide, is present in step b) at a final concentration of about 2 mM. In one embodiment, the oxidizing agent, preferably hydrogen peroxide, is present in step b) at a final concentration of about 4 mM.

In one embodiment, the oxidizing agent is a hydrogen peroxide-generating system, preferably comprising an oxidase preferably glucose oxidase. In one embodiment, the oxidase, is present in step b) at a final concentration suitable for carrying out the method including but not limited to a final concentration of about 0.25 u/mL to 10 U/mL, or about 0.5 U/mL to 5 U/mL, or about 1 U/mL to 4.5 U/mL, or about 2 U/mL to about 4 U/mL. In one embodiment, the oxidase is present in step b) at a final concentration of about 1 U/mL. In one embodiment, the oxidase is present in step b) at a final concentration of about 2 U/mL. In one embodiment, the oxidase is present in step b) at a final concentration of about 4 U/mL. In one embodiment, glucose in step b) is present at a final concentration suitable for carrying out the method, preferably a final concentration of about 1 mM to 20 mM, or about 5 mM to about 15 mM, or about 7 mM to 10 mM. In one embodiment, glucose in step b) is present at a final concentration about 10 mM.

In one embodiment, the catalyst is a horseradish peroxidase. In one embodiment, the catalyst, preferably horseradish peroxidase, is present in step b) at a final concentration suitable for carrying out the method including but not limited to a final concentration of about 0.5 u/mL to 10 U/mL, or about 1 U/mL to 5 U/mL, or about 2 U/mL to 4.5 U/mL. In one embodiment, the catalyst, preferably horseradish peroxidase, is present in step b) at a final concentration of about 4.5 U/mL.

In one embodiment, the detection reagent in step b) is present at a final concentration suitable for carrying out the method including but not limited to a final concentration of about 0.1 mM to 5 mM, or about 0.2 mM to 3 mM, or about 0.5 mM to 2 mM. In one embodiment, the detection reagent in step b) is present at a final concentration of about 0.5 mM or about 2 mM.

In one embodiment, the oxidizing reagent is not air oxygen.

In this aspect of the method, step b) may comprise contacting the sample of step a) with p-hydroxyhippuric acid or a salt thereof, the detection reagent, the oxidizing agent or the catalyst simultaneously and optionally incubating the detection mixture.

Alternatively, step b) may comprise contacting the sample of step a) with p-hydroxyhippuric acid or the salt thereof, the detection reagent, the oxidizing agent or the catalyst separately and optionally incubating the detection mixture.

In one embodiment, step b) may comprise contacting the sample of step a) with p-hydroxyhippuric acid or the salt thereof; optionally incubating; followed by adding, preferably simultaneously, the detection reagent, the catalyst and the oxidizing agent.

In one embodiment, step b) may comprise contacting the sample of step a) with p-hydroxyhippuric acid or the salt thereof; optionally incubating; and followed by adding the detection reagent and the catalyst simultaneously, and by further adding the oxidizing agent. Alternatively, the catalyst and oxidizing agent may be added simultaneously, and the detection reagent may be added separately.

In one embodiment, step b) may comprise contacting the sample of step a) with p-hydroxyhippuric acid or the salt thereof and the detection reagent; optionally incubating, followed by adding the catalyst and the oxidizing agent. In a specific embodiment p-hydroxyhippuric acid or the salt thereof and the detection reagent may be added simultaneously, and the catalyst and the oxidizing agent may be added separately or simultaneously. In a further specific embodiment, p-hydroxyhippuric acid or the salt thereof and the detection reagent may be added separately, and the catalyst and the oxidizing agent may be added simultaneously.

In a preferred embodiment, step b) may comprise contacting the sample of step a) with p-hydroxyhippuric acid or the salt thereof, the detection reagent, and the catalyst; optionally incubating; followed by adding the oxidizing agent. In a specific embodiment, p-hydroxyhippuric acid or the salt thereof, the detection reagent, and the catalyst may be added separately. In a specific embodiment, p-hydroxyhippuric acid or the salt thereof, the detection reagent, and the catalyst may be added simultaneously. In a specific embodiment, p-hydroxyhippuric acid or the salt thereof and the detection reagent may be added simultaneously followed by adding the catalyst. In a specific embodiment, p-hydroxyhippuric acid or the salt thereof and the catalyst may be added separately followed by adding the detection reagent.

The invention further relates to a kit comprising p-hydroxyhippuric acid or a salt thereof, a detection reagent, a catalyst, and an oxidizing agent, e.g., for contacting a sample to be tested for the presence or absence of a microorganism having hippuricase activity.

In one embodiment, the detection reagent is a reagent for phenol detection as described herein, e.g., selected from the group consisting of 4-aminoantipyrine or a salt thereof, 3-methyl-2-benzothiazolinone hydrazone or a salt thereof, and N-methyl-N-phenyl-3-sulfophenylenediamine or a salt thereof. In a preferred embodiment, the detection reagent is selected from the group consisting of 4-aminoantipyrine or a salt thereof and 3-methyl-2-benzothiazolinone hydrazone or a salt thereof. In a particularly preferred embodiment, the detection reagent is 4-aminoantipyrine or a salt thereof. In yet a further preferred embodiment, the catalyst is a peroxidase, preferably horseradish peroxidase.

In yet a further preferred embodiment, the oxidizing agent is hydrogen peroxide and/or a hydrogen peroxide-generating system wherein preferably the hydrogen peroxide-generating system is an oxidase and a substrate, wherein more preferably the oxidase is a glucose oxidase, and/or the substrate is glucose.

In a further embodiment, the kit further comprises one or more auxiliary agents such as a preservative, an antioxidant, water, stabilizer or buffer.

In one embodiment of the kit of the invention, one or more of the reagents of the kit such as p-hydroxyhippuric acid or the salt thereof, the detection reagent, the catalyst, the oxidizing agent and/or the auxiliary agent is/are present in liquid form, e.g., as solution, suspension, emulsion or in oily or aqueous vehicles.

In a preferred embodiment of the kit of the invention, one or more of the reagents of the kit such a p-hydroxyhippuric acid or the salt thereof, the detection reagent, the catalyst, the oxidizing agent and/or the auxiliary agent is/are present in solid form, e.g., as a powder, granules, a tablet or freeze-dried, and the kit optionally comprises one or more further container(s) comprising water or a buffer such as a phosphate buffer for reconstitution. In a preferred embodiment, the catalyst and/or the oxidase are freeze-dried. In a further preferred embodiment, p-hydroxyhippuric acid is solid.

In one embodiment of the invention, the kit comprises a first container comprising p-hydroxyhippuric acid, a second container comprising the detection reagent, a third container comprising the catalyst, and a fourth container comprising the oxidizing agent, and optionally instructions for contacting a sample to be tested for comprising the microorganism with the p-hydroxyhippuric acid, the detection reagent, the catalyst, and the oxidizing agent.

In another embodiment, the kit comprises a first container comprising p-hydroxyhippuric acid, a second container comprising the detection reagent and the catalyst, and a third container comprising the oxidizing agent, and optionally instructions for contacting a sample to be tested for comprising the microorganism with the p-hydroxyhippuric acid, the detection reagent, the catalyst, and the oxidizing agent.

In another embodiment, the kit comprises a first container comprising p-hydroxyhippuric acid and a second container comprising the detection reagent, the catalyst, and the oxidizing agent, and optionally instructions for contacting a sample to be tested for comprising the microorganism with the p-hydroxyhippuric acid, the detection reagent, the catalyst, and the oxidizing agent.

In yet another embodiment, the kit comprises a first container comprising p-hydroxyhippuric acid and the detection reagent, a second container comprising the catalyst, and a third container comprising the oxidizing agent, and optionally instructions for contacting a sample to be tested for comprising the microorganism with the p-hydroxyhippuric acid, the detection reagent, the catalyst, and the oxidizing agent.

In yet another embodiment, the kit comprises a first container comprising p-hydroxyhippuric acid and the detection reagent, and a second container comprising the catalyst and the oxidizing agent, and optionally instructions for contacting a sample to be tested for comprising the microorganism with the p-hydroxyhippuric acid, the detection reagent, the catalyst, and the oxidizing agent.

In yet another embodiment, the kit comprises a first container comprising p-hydroxyhippuric acid, a second container comprising the detection reagent, and a third container comprising the catalyst and the oxidizing agent, and optionally instructions for contacting a sample to be tested for comprising the microorganism with the p-hydroxyhippuric acid, the detection reagent, the catalyst, and the oxidizing agent.

In yet another embodiment, the kit comprises a first container comprising p-hydroxyhippuric acid, the detection reagent, and the catalyst, and a second container comprising the oxidizing agent, and an oxidizing agent, and optionally instructions for contacting a sample to be tested for comprising the microorganism with the p-hydroxyhippuric acid, the detection reagent, the catalyst, and the oxidizing agent.

In yet another embodiment, the kit comprises a container comprising p-hydroxyhippuric acid, the detection reagent, the catalyst, and the oxidizing agent, and optionally instructions for contacting a sample to be tested for comprising the microorganism with the p-hydroxyhippuric acid, the detection reagent, the catalyst and the oxidizing agent.

In a further embodiment, the kit further comprises a container comprising a culture medium suitable for culturing and/or pre-enrichment of the sample to be tested as described herein.

In a preferred embodiment of the kit of the invention, the container is a vial, ampoule, or a membrane such aa a nitrocellulose membrane. The membrane may be in the form of a strip or a disk. The membrane may comprise one or more of the reagents of the kit as described herein. For example, the membrane may comprise, e.g., may be saturated, with p-hydroxyhippuric acid or a salt thereof. Alternatively, the membrane may comprise, e.g., may be saturated with p-hydroxyhippuric acid or a salt thereof and the detection reagent.

In a further embodiment, the kit further comprises a container suitable for one or more pre-treatment steps, e.g., for culturing and/or pre-enrichment of the sample to be tested as described herein.

The invention further relates to the use, preferably ex vivo or in vitro, of the kit for detection of a microorganism having hippuricase activity, preferably hippuricase activity of an endogenous hippuricase enzyme, preferably in a sample.

In one embodiment, the microorganism is a bacterium. In a preferred embodiment, the bacterium comprises a catalase, i.e., the bacterium is catalase positive. In a further preferred embodiment, the microorganism is a bacterium preferably from Campylobacter species, e.g., Campylobacter jejuni or Campylobacter avium; Gardnerella species, e.g. Gardnerella vaginalis, Streptococcus species, e.g., Streptococcus group B (Streptococcus agalactiae), preferably bovine Streptococcus group B; Staphylococcus species, e.g., Staphylococcus aureus; Legionella species and/or Listeria species, e.g., Listeria monocytogenes. In a more preferred embodiment, the microorganism is from Campylobacter species, preferably Campylobacter jejuni. In a more preferred embodiment, the microorganism is from Streptococcus species, preferably Streptococcus agalactiae.

In a further embodiment, the sample is from a subject such as an animal. The animal may be an animal that lives in close relationship with a human such a farm animal or a pet. Examples of animals are dogs, cats, birds, cattle, sheep swine, goat or poultry. In a preferred embodiment, the subject is a human. The sample from the subject may be derived from a body fluid, such as blood, serum, bone marrow, salvia, vaginal fluid, urine, cerebrospinal fluid, placental or umbilical cord blood, lymphoid fluid, feces, and/or from a tissue of the subject.

In a further embodiment, the sample is from environment. For example, the sample might be from water, e.g., drinking water; dung; stables such as stables from farm animals; dust from industrial facilities such as floor, devices, and facilities; sand or other sediments; soil or industrial waste.

In a further preferred embodiment, the sample is from a product intended for human consumption such as food. Examples are meat, e.g., from poultry, cattle, sheep, swine and/or goat; dairy product, e.g., milk (raw or pasteurized); vegetables; fish, e.g., shellfish; or fruits.

In one embodiment, the sample comprises a microorganism.

The invention further relates to the use, preferably ex vivo or in vitro, of the kit as described above for discriminating a microorganism having hippuricase activity from a microorganism without hippuricase activity, preferably in a sample.

In one embodiment, the microorganism is a bacterium. In a preferred embodiment, the bacterium comprises a catalase, i.e., the bacterium is catalase positive. In a further preferred embodiment, the microorganism is a bacterium preferably from Campylobacter species, e.g., Campylobacter jejuni or Campylobacter avium; Gardnerella species, e.g. Gardnerella vaginalis, Streptococcus species, e.g., Streptococcus group B (Streptococcus agalactiae), preferably bovine Streptococcus group B; Staphylococcus species, e.g., Staphylococcus aureus; Legionella species and/or Listeria species, e.g., Listeria monocytogenes. In a more preferred embodiment, the microorganism is from Campylobacter species, preferably Campylobacter jejuni. In a more preferred embodiment, the microorganism is from Streptococcus species, preferably Streptococcus agalactiae.

In a further embodiment, the sample is from a subject such as an animal. The animal may be an animal that lives in close relationship with a human such a farm animal or a pet. Examples of animals are dogs, cats, birds, cattle, sheep swine, goat or poultry. In a preferred embodiment, the subject is a human. The sample may be from a body fluid, such as blood, serum, bone marrow, salvia, vaginal fluid, urine, cerebrospinal fluid, placental or umbilical cord blood, lymphoid fluid, feces, and/or from a tissue of the subject.

In a further embodiment, the sample is from environmental. For example, the environmental sample might be water, e.g., drinking water; dung; samples derived from stables such as stables from farm animals; dust and/or samples derived from industrial facilities such as floor, devices, and facilities; sand or other sediments; soil or industrial waste.

In a further preferred embodiment, the sample is from a product intended for human consumption such as food. Examples are meat, e.g., from poultry, cattle, sheep, swine and/or goat; dairy product, e.g., milk (raw or pasteurized); vegetables; fish, e.g., shellfish; or fruits.

In one embodiment, the sample comprises a microorganism.

The invention further relates to the use, preferably ex vivo or in vitro, of the kit in the method as described herein.

The invention further relates to a method of detection of a microorganism having hippuricase activity, preferably hippuricase activity of an endogenous hippuricase enzyme, comprising the use of the kit of the invention.

The invention further relates to a method for discriminating a microorganism having hippuricase activity from a microorganism without hippuricase activity, comprising the use of the kit of the invention.

The invention further relates to a method for detecting a microorganism having hippuricase activity, comprising the use of the kit of the invention.

The invention further relates to a method of treating an infection by a microorganism, preferably in a subject in need thereof, comprising:
a) providing a sample to be tested from the subject,
b) contacting and optionally incubating the sample from step a) with p-hydroxyhippuric acid or a salt thereof, a detection reagent, preferably a reagent for phenol detection as described herein, and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent,
c) determining in the detection mixture of step b) the presence or absence of the detectable compound wherein the presence of the detectable compound indicates the presence of the microorganism having hippuricase activity, and
d) administering an antibiotic to the subject if the presence of a detectable compound has been determined in step c).

The present invention is further characterized by the following items:
1. A method of detecting a microorganism having hippuricase activity comprising:
a) providing a sample to be tested, wherein the sample preferably comprises a microorganism,
b) contacting and optionally incubating the sample from step a) with p-hydroxyhippuric acid or a salt thereof, a detection reagent, preferably a reagent for phenol detection, and an oxidizing reagent to obtain a detection mixture wherein, if the microorganism with hippuricase activity is present, a detectable compound is produced, wherein preferably the detectable compound is produced by oxidative coupling of p-hydroxybenzoic acid with the detection reagent in the presence of the oxidizing reagent, and
c) determining in the detection mixture of step b) the presence or absence of the detectable compound wherein the presence of the detectable compound indicates the presence of the microorganism having hippuricase activity.
2. The method of item 1 wherein the detection reagent is a compound which modifies p-hydroxybenzoic acid to produce a chromogenic detectable compound, preferably a compound which reacts via oxidative coupling to produce a chromogenic detectable compound, more preferably wherein the detection reagent is selected from the group consisting of 4-aminoantipyrine or a salt thereof and 3-methyl-2-benzothiazolinone hydrazone or a salt thereof.
3. The method of item 1 or 2 wherein the oxidizing reagent comprises an oxidizing agent and a catalyst.
4. The method of item 3 wherein in step b) the sample is contacted with p-hydroxyhippuric acid, or the salt thereof followed by adding the detection reagent, the catalyst, and the oxidizing agent.
5. The method of item 3 or 4 wherein in step b) the sample is contacted with p-hydroxyhippuric acid, or the salt thereof followed by simultaneous addition of the detection reagent and the catalyst, and further addition of the oxidizing agent.
6. The method of item 3 wherein in step b) the sample is contacted with p-hydroxyhippuric acid or the salt thereof and the detection reagent, preferably simultaneously, followed by adding the catalyst and the oxidizing agent.
7. The method of item 3 or 6 wherein in step b) the sample is contacted with p-hydroxyhippuric acid or the salt thereof and the detection reagent simultaneously followed by adding the catalyst and the oxidizing agent.
8. The method of item 3 wherein in step b) the sample is contacted with p-hydroxyhippuric acid or the salt thereof, the detection reagent and the catalyst, preferably simultaneously, followed by adding the oxidizing agent.
9. The method of any one of the items 3 to 8 wherein the oxidizing agent is hydrogen peroxide and/or a hydrogen peroxide-generating system wherein preferably the hydrogen peroxide-generating system is an oxidase and a substrate, wherein more preferably the oxidase is a glucose oxidase, and/or the substrate is glucose.
10. The method of any one of items 3 to 9 wherein the catalyst is a peroxidase, preferably horseradish peroxidase.
11. The method of item 1 or 2 wherein the oxidizing reagent comprises a halogen, such as a halogen salt, preferably a periodate salt, more preferably sodium periodate.
12. The method of item 1, 2 or 11 wherein in step b) the sample is contacted with the p-hydroxyhippuric acid, or the salt thereof followed by the detection reagent and the oxidizing reagent, preferably simultaneously.
13. The method of any one of the preceding items wherein step b) and step c) are direct consecutive steps.
14. The method of any one of the preceding items wherein in step c) determining in the detection mixture of step b) the presence or absence of the detectable compound comprises detecting a compound-specific absorbance wherein optionally detecting the compound-specific absorbance is qualitatively, such as by visual inspection, and/or quantitatively, such as by measuring the absorbance.
15. The method of any one of the preceding items wherein the microorganism is a bacterium preferably from Campylobacter species, Gardnerella species, Streptococcus species, Staphylococcus species, Legionella species and/or Listeria species.
16. The method of any one of the preceding items wherein the microorganism is from Campylobacter species and is selected from the group consisting of Campylobacter jejuni and Campylobacter avium; or wherein the microorganism is from Gardnerella species and is Gardnerella vaginalis; or wherein the microorganism is from Streptococcus species and is selected from the group consisting of Streptococcus group B, more preferably bovine Streptococcus group B, and Streptococcus agalactiae; or wherein the microorganism is from Staphylococcus species and is Staphylococcus aureus; or wherein the microorganism is from Listeria species and is Listeria monocytogenes.
17. The method of any one of the preceding items wherein step a) further comprises a culturing step, optionally wherein a pre-enrichment step precedes the culturing step.
18. The method of any of the preceding items wherein step a) further comprises a culturing step, wherein optionally a pre-enrichment step precedes the culturing step, wherein the culturing step and, optionally the pre-enrichment step, comprises preferably culturing the sample under microaerobic atmosphere, containing preferably oxygen in a range of about 2% to about 10%, and with preferably, a temperature from about 37° C. to about 42° C. and wherein preferably the microorganism is of Campylobacter species.
19. The method of item 17 or 18 wherein the culturing step is performed as described in ISO 10272-1:2017 and/or ISO 11133:2014.
20. The method of any one of the preceding items wherein the sample in step a) is from a subject, preferably human and preferably from feces and/or a body fluid.
21. The method of item 20 wherein the method further comprises:
d) administering an antibiotic to the subject if the presence of a detectable compound has been determined in step c).
22. The method of any one of items 1 to 19 wherein the sample in step a) is from food, preferably from meat and/or milk.
23. The method of any one of items 1 to 19 wherein the sample in step a) is from environment, preferably from water, dung, dust and/or industrial waste.
24. A kit for detecting a microorganism with hippuricase activity comprising:
a) a first container comprising p-hydroxyhippuric acid, a second container comprising a detection reagent, and a third container comprising an oxidizing reagent, or
b) a container comprising p-hydroxyhippuric acid, a detection reagent, and an oxidizing reagent, or
c) a first container comprising p-hydroxyhippuric acid and a second container comprising a detection reagent and an oxidizing reagent, or
d) a first container comprising p-hydroxyhippuric acid and a detection reagent and a second container comprising an oxidizing reagent, and
(e) optionally instructions for contacting a sample to be tested for comprising the microorganism with p-hydroxyhippuric acid, the detection reagent, and the oxidizing reagent.
25. The kit of item 24 wherein the detection reagent is selected from the group consisting of 4-aminoantipyrine or a salt thereof and 3-methyl-2-benzothiazolinone hydrazone or a salt thereof
26. The kit of item 24 or 25 wherein the oxidizing reagent is a halogen, such as a halogen salt, preferably a periodate salt, more preferably sodium periodate.
27. The kit of any one of items 24 to 26 wherein one or more of the containers further comprises an auxiliary agent.
28. The kit of any one of items 24 to 27 wherein the kit further comprises a container comprising a culture medium.
29. A kit for detecting a microorganism with hippuricase activity comprising:
a) a first container comprising p-hydroxyhippuric acid, a second container comprising a detection reagent, a third container comprising a catalyst, and a fourth container comprising an oxidizing agent, or
b) a first container comprising p-hydroxyhippuric acid, a second container comprising a detection reagent and a catalyst, and a third container comprising an oxidizing agent, or
c) a first container comprising p-hydroxyhippuric acid and a second container comprising a detection reagent, a catalyst, and an oxidizing agent, or
d) a first container comprising p-hydroxyhippuric acid and a detection reagent, a second container comprising a catalyst, and a third container comprising an oxidizing agent, or
e) a first container comprising p-hydroxyhippuric acid and a detection reagent, and a second container comprising a catalyst, and an oxidizing agent, or
f) a first container comprising p-hydroxyhippuric acid, a second container comprising a detection reagent, and a third container comprising a catalyst and an oxidizing agent, or
(g) a first container comprising p-hydroxyhippuric acid, a detection reagent, and a catalyst, and a second container comprising an oxidizing agent, or
(h) a container comprising p-hydroxyhippuric acid, a detection reagent, a catalyst, and an oxidizing agent, and
i) optionally instructions for contacting a sample to be tested for comprising the microorganism with the p-hydroxyhippuric acid, the detection reagent, the catalyst and the oxidizing agent.
30. The kit of item 29 wherein the detection reagent is selected from the group consisting of 4-aminoantipyrine or a salt thereof and 3-methyl-2-benzothiazolinone hydrazone or a salt thereof.
31. The kit of item 29 or 30 wherein the oxidizing agent is hydrogen peroxide and/or a hydrogen peroxide-generating system wherein preferably the hydrogen peroxide-generating system is an oxidase and a substrate, wherein more preferably the oxidase is a glucose oxidase, and/or the substrate is glucose.
32. The kit of any one of items 29 to 31 wherein the catalyst is a peroxidase, preferably horseradish peroxidase.
33. The kit of any one of items 29 to 32 wherein one or more of the containers further comprises an auxiliary agent.
34. The kit of any one of items 29 to 33 wherein the kit further comprises a container comprising a culture medium.
35. Use of the kit of any one of items 24 to 34 for detection of a microorganism having hippuricase activity, preferably hippuricase activity of an endogenous hippuricase enzyme.
36. Use of the kit of any one of items 24 to 34 for discriminating a microorganism having hippuricase activity from a microorganism without hippuricase activity.
37. The use of item 35 or 36 wherein the microorganism is a culturable microorganism.
38. The use of any one of items 35 to 37 wherein the microorganism is a bacterium, preferably from Campylobacter species, Gardnerella species, Streptococcus species, Staphylococcus species, Legionella species and/or Listeria species.
39. The use of any one of items 35 to 38 wherein the microorganism is from Campylobacter species and is selected from the group consisting of Campylobacter jejuni and Campylobacter avium; or wherein the microorganism is from Gardnerella species and is Gardnerella vaginalis; or wherein the microorganism is from Streptococcus species and is selected from the group consisting of Streptococcus group B, preferably bovine Streptococcus group B, and Streptococcus agalactiae; or wherein the microorganism is from Staphylococcus species and is Staphylococcus aureus; or wherein the microorganism is from Listeria species and is Listeria monocytogenes.
40. The use of any one of items 35 to 39 wherein the use is ex vivo, in vitro or the detection of the microorganism is in a sample.
40. Use of the kit of any one of items 24 to 28 in the method of any one of items 1, 2 or 11 to 23.
41. Use of the kit of any one of items 29 to 34 in the method of any one of items 3 to 10 or 13 to 23.

### EXAMPLES

### Example 1: Detection of Campylobacter jejuni

Campylobacter jejuni DSM 4688 (C. j., catalase positive), Eschericha coli (E. coli, catalase positive) and Campylobacter coli DSM 4689 (C. c., catalase positive), Streptococcus agalactiae ATCC®12386 (S. a., catalase negative) and Streptococcus pyogenes ATCC®19615 (S. p., catalase negative) were cultivated on Columbia Agar with sheep blood (Thermofischer PB5039A) at 37° C. for 48 h, both Campylobacter strains were cultivated under microaerophilic atmosphere (Oxoid, Campylgen CN0025A).

Biomass of colonies were suspended in sterile phosphate buffer (100 mM, pH 7.4) and the optical density OD was determined and set to OD₆₀₀ of 0.9.

Two hundred μL of cell suspensions were mixed with 25 μL of 4-hydroxyhippuric acid (4-HHA, Bachem, 4005059) stock solution, prepared in 100 mM phosphate buffer pH adjusted to 7.4 in a microtiter plate (SpektraMax M5, Molecular Devices) and incubated for 70 minutes at 37° C. After incubation, 20 μL of a prepared mixture (12.5×) 4-aminoantipyrine (4-AAP, Merck 06800) and peroxidase (HRP, Merck 77332) and 5 μL of 100 mM H₂O₂ were added. The absorbance at 505 nm was recorded with a plate reader at room temperature. Final concentrations for the assay were 5 mM, 10 mM and 20 mM 4-HHA, 0.5 mM 4-AAP, 4.5 U/mL HRP and 2 mM H₂O₂.

The samples comprising Campylobacter coli, Eschericha coli and the sterile control remained colorless or slightly yellowish while the samples comprising Campylobacter jejuni showed a robust red to pink color after a few minutes.

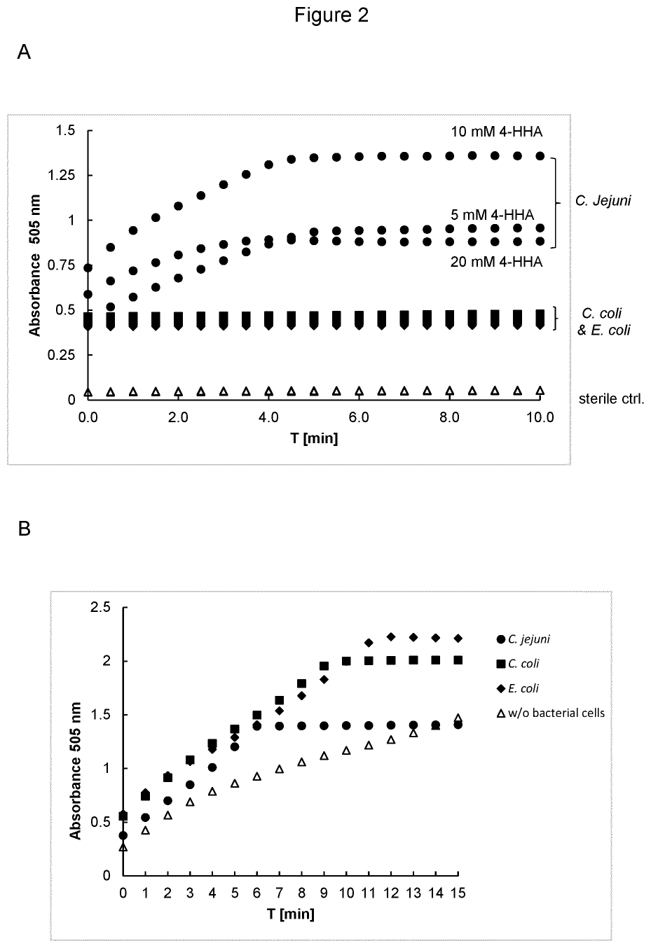

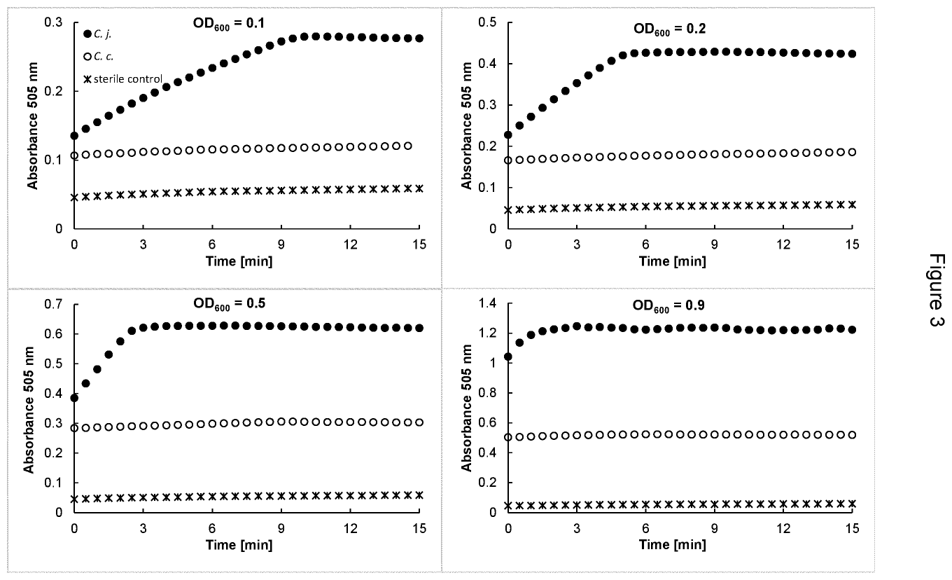

The formation of the quinoneimine compound produced by oxidative coupling of p-hydroxybenzoic acid with 4-aminoantipyrine and peroxidase in the presence of the hydrogen peroxide was determined. The results in FIG. 2A show a significant increase of the absorbance at 505 nm with a plateau reached after about 5 minutes. No background coloration has been observed with the hippuricase negative bacteria strain demonstrating the high specificity of the method for hippuricase positive bacteria. Further, a final concentration of 10 mM 4-HHA showed the best performance. It was also found that hydrogen peroxide is preferably added after incubating the bacteria with 4-HHA in order to yield optimal color results which are visible by eye (data not shown).

In a catalase control experiment, biomass of colonies was suspended in sterile phosphate buffer (100 mM, pH 7.4) and the optical density OD was determined and then serially diluted with the same buffer to an OD₆₀₀ of 0.4 to 0.5.

One hundred to 150 μL of cell suspension were mixed with 50 to 100 μL of 100 mM phosphate puffer pH 7.4 and 50 μL of the prepared mixture (5×) 4-aminoantipyrine (4-AAP, Merck 06800, 2.5 mM), peroxidase (HRP, Merck 77332, 22.5 U/mL) and 4-hydroxybenzoic acid (4-HBA, Merck H20059, 5 mM, dissolved in 100 mM phosphate puffer pH 7.4) and 5 μL of 50 mM H₂O₂ were added. The absorbance at 505 nm was recorded with a plate reader (SpektraMax M5, Molecular Devices) at room temperature. Final concentrations for the assay are 1 mM 4-HBA, 0.5 mM 4-AAP, 4.5 U/mL HRP and 1 mM H₂O₂. (FIG. 2B)

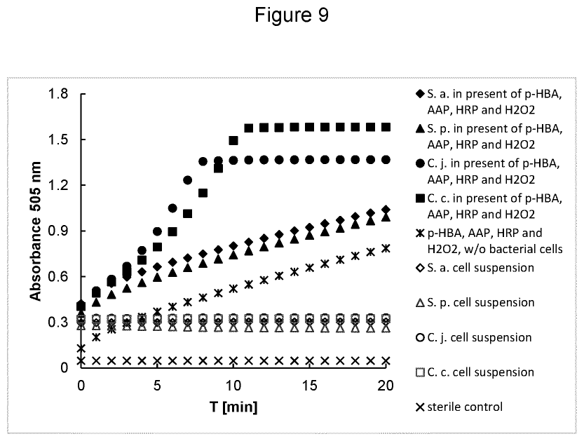

Two hundred μL of cell suspension were mixed with 20 μL of 100 mM phosphate puffer pH 7.4, 5 μL 50 mM and p-4-hydroxybenzoic acid (p-HBA, Merck H20059 dissolved in 100 mM phosphate puffer pH 7.4), 20 μL of mixture 4-aminoantipyridine (Merck 06800, 6.25 mM) and horseradish peroxidase (Merck 77332, 56.3 U/mL), followed by the addition of 5 μL of 50 mM H₂O₂. The absorbance at 505 nm was recorded with a plate reader (SpektraMax M5, Molecular Devices) at room temperature. Final concentrations for the assay are 1 mM p-4-HBA, 0.5 mM 4-AAP, 4.5 U/mL HRP and 1 mM H₂O₂. (FIG. 9)

The formation of the quinoneimine compound produced by oxidative coupling of p-hydroxybenzoic acid with 4-aminoantipyrine was determined by measuring the absorbance at 505 nm (FIG. 2B and FIG. 9). While absorbance in the control without cells continued to increase for 15 minutes, the reaction stopped after about 5 minutes in the catalase-positive samples (Campylobacter). In the catalase negative samples (Streptococcus), the reaction kinetics are similar to the reaction without cell suspension. These results show that catalase most likely degraded the added hydrogen peroxide which caused a stop of the reaction of 4-AAP/HRP with p-4-HBA. However, the coloration plateau in the hippuricase assay was reached in a few minutes (FIG. 2A) showing that the hippuricase reaction is sufficient fast and qualifies as a detecting method in microorganisms despite the presence of catalase.

### Example 2: Detection Campylobacter jejuni Using HRP and H₂O₂ as Oxidizing Reagent

Campylobacter jejuni DSM 4688 (C. j., hippuricase positive) and Campylobacter coli DSM 4689 (C. c., hippuricase negative) were cultivated on Columbia Agar with sheep blood (Thermofischer PB5039A) under microaerophilic atmosphere (Oxoid, Campylgen CN0025A) at 37° C. for 48 h.

Biomass of colonies were suspended in sterile 100 mM phosphate buffer pH 7.4 and the optical density OD₆₀₀ was set to 0.9 and then serially diluted with the same buffer to an OD₆₀₀ of 0.5, 0.2 and 0.1.

Two hundred μL of cell suspensions were mixed with 25 μL of 100 mM 4-hydroxyhippuric acid (4-HHA, Bachem 4005059) prepared in 100 mM phosphate buffer pH adjusted to 7.4) in a microtiter plate and incubated for 1 h at 37° C. After incubation, 20 μL of the prepared mixture (12.5×) 4-aminoantipyrine (4-AAP, Merck 06800) and peroxidase (HRP, Merck 77332), 5 μL of 100 mM H₂O₂ were added. The absorbance at 505 nm was recorded with a plate reader (SpektraMax M5, Molecular Devices) at room temperature. Final concentrations for the assay were 10 mM 4-HHA, 0.5 mM 4-AAP, 4.5 U/mL HRP and 2 mM H₂O₂.

The samples comprising Campylobacter coli or sterile control remained colorless or slightly yellowish at all OD₆₀₀ values while the samples comprising Campylobacter jejuni showed a robust red to pink color over a broad range of OD₆₀₀ values which gradual increased in intensity from an OD₆₀₀ value of 0.1 to an OD₆₀₀ value 0.9. The red/pink color was even visible by eye in the sample with an OD₆₀₀ value of 0.1 corresponding to a cell concentration of about 2×10⁶ CFU/mL (Krüger et al. PLoS One. 2014 Feb. 5; 9(2):e88108).

The formation of the quinoneimine compound produced by oxidative coupling of p-hydroxybenzoic acid with 4-aminoantipyrine in the presence of the hydrogen peroxide was determined by measuring the absorbance at 505 nm. The results in FIG. 3 show a significant increase of the absorbance already after a few minutes with a plateau reached, e.g., after about 10 minutes at an OD₆₀₀ value of 0.1 demonstrating that the method has a high sensitivity and allows for fast detection of bacteria at low cell density. No background coloration has been observed with the hippuricase negative bacteria strain demonstrating the high specificity of the method for hippuricase positive bacteria.

### Example 3 Detection Campylobacter jejuni Using H₂O₂ In-Situ-Produced Per Glucose/Glucose Oxidase

Campylobacter jejuni DSM 4688 (C. j., hippuricase positive) and Campylobacter coli DSM 4689 (C. c., hippuricase negative) were cultivated on Columbia Agar with sheep blood (Thermofischer PB5039A) under microaerophilic atmosphere (Oxoid, Campylgen CN0025A) at 37° C. for 48 h.

Biomass of colonies were suspended in sterile phosphate buffer (100 mM, pH 7.4) and the optical density OD was set to 0.9 and then serially diluted with the same buffer to an OD₆₀₀ of 0.5, 0.2 and 0.1.

Two hundred μL of cell suspension were mixed with 25 μL of 100 mM 4-hydroxyhippuric acid (4-HHA, Bachem 4005059, dissolved in 100 mM phosphate puffer pH 7.4) in microtiter plate and incubated for 1 h at 37° C. After incubation, 10 μL of the prepared mixture (25×) 4-aminoantipyrine (4-AAP, Merck 06800) and peroxidase (HRP, Merck 77332), 10 μL of 250 mM glucose (Merck, G8270) and 5 μL of glucose oxidase (GOD, Merck G2133) 50 U/mL were added. The absorbance at 505 nm was recorded with a plate reader. Final concentration of 4-HHA was 10 mM, 4-AAP was 0.5 mM, HRP was 4.5 U/mL, glucose was 10 mM and GOD was 1 U/mL.

The samples comprising Campylobacter coli remained colorless or slightly yellowish for all OD₆₀₀ values while in the samples comprising Campylobacter jejuni showed a robust red to pink color over a broad range of OD₆₀₀ values which gradual increase in intensity from an OD₆₀₀ value of 0.1 to an OD₆₀₀ value 0.9 as determined by visual inspection. The red color was even visible by eye in the sample with an OD₆₀₀ value of 0.1 corresponding to a cell concentration of about 2×10⁶ CFU/mL (Krüger et al. PLoS One. 2014 Feb. 5; 9(2):e88108).

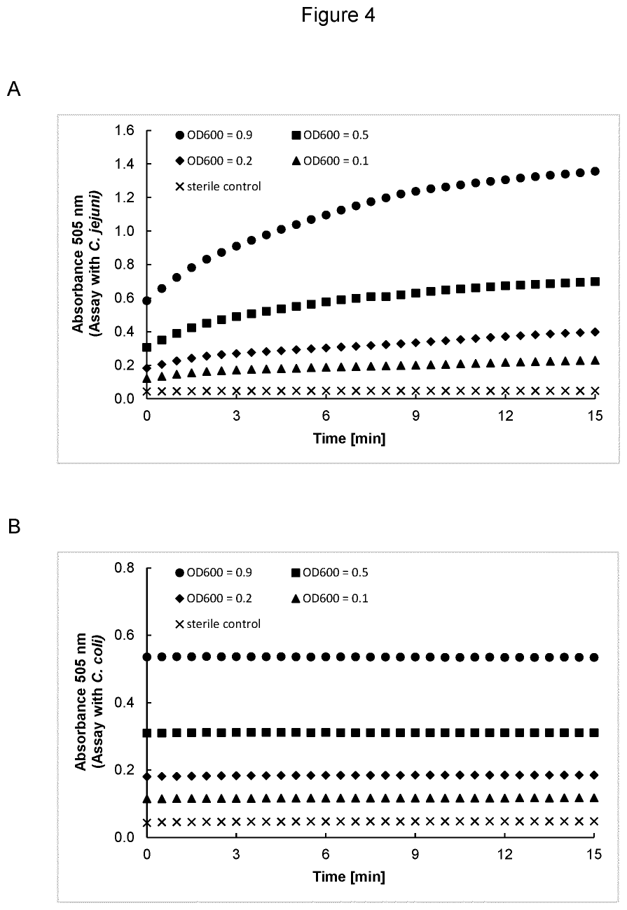

The formation of the quinoneimine compound produced by oxidative coupling of p-hydroxybenzoic acid with 4-aminoantipyrine in the presence of the hydrogen peroxide generating system glucose/glucose oxidase was determined by measuring the absorbance at 505 nm. The results in FIG. 4 compare absorbance signal development over time of A: C. jejuni and B: C. coli at similar cell densities. This example confirms that the method is also sensitive and specific with a hydrogen peroxide generating system.

### Example 4 Detection of Campylobacter jejuni—Comparison with Ninhydrin Method

a) Inventive method
b) Hippurate disk (Remel, Thermo Scientific, R21085)
c) Hippurate strips kit (Millipore 01869)

Campylobacter jejuni DSM 4688 (C. j., hippuricase positive) and Campylobacter coli DSM 4689 (C. c., hippuricase negative) were cultivated on Columbia Agar with sheep blood (Thermofischer PB5039A) under microaerophilic atmosphere (Oxoid, Campylgen CN0025A) at 37° C. for 48 h.

Biomass of colonies were suspended, a) 0.2 mL sterile 100 mM phosphate buffer pH 7.4; b) 0.1 mL sterile water (as recommended in manual), c) 0.5 mL saline solution (as recommended in manual). To ensure a proper comparability, the optical density OD600 of all cell suspensions were set to 0.2. In method a) 25 μL of 100 mM 4-hydroxyhippuric acid (4-HHA, Bachem 4005059 prepared in 100 mM phosphate buffer pH adjusted to 7.4) was added to cell suspensions. In methods b) and c), Hippurate disks and Hippurate strips were dropped in cell suspensions as recommended in manual. To ensure a proper comparability, all samples were incubated at 37° C. for 1 h.

After incubation, 10 μL of the prepared mixture (25×) 4-aminoantipyrine (4-AAP, Merck 06800) and peroxidase (HRP, Merck 77332), followed with 5 μL of 100 mM H₂O₂ were added in samples of method a). Final concentrations for the assay were 10 mM 4-HHA, 0.5 mM 4-AAP, 4.5 U/mL HRP and 2 mM H₂O₂. A significant pink color was visible by eye in sample comprising Campylobacter jejuni in few minutes at room temperature, while sample comprising Campylobacter coli or sterile control remained slightly yellowish.

After incubation, ninhydrin reagent was added to samples of method (b), the samples were reincubated at 37° C. for 30 min (as recommended in manual). A pale blue color is visible in sample comprising Campylobacter jejuni while sample comprising Campylobacter coli or sterile control remained slightly yellowish.

After incubation, ninhydrin reagent was added to samples of method (c), the samples were reincubated at room temperature for 10 min (as recommended in manual). No color change was observed in all samples comprising Campylobacter jejuni, Campylobacter coli and sterile control.

The competitive advantages of the inventive method are: i) no extended incubation at 37° C. for coloration, ii) shorter assay time, iii) no corrosive organic solution, iv) sensitive and high specific.

### Example 5: Detection Campylobacter jejuni Using Sodium Periodate as Oxidizing Reagent

Campylobacter jejuni DSM 4688 (C. j., hippuricase positive) and Campylobacter coli DSM 4689 (C. c., hippuricase negative) were cultivated on Columbia Agar with sheep blood (Thermofischer PB5039A) under microaerophilic atmosphere (Oxoid, Campylgen CN0025A) at 37° C. for 48 h.

Biomass of colonies were suspended in sterile 100 mM phosphate buffer pH 7.4 and the optical density OD₆₀₀ was set to 1.1 and then serially diluted with the same buffer to an OD₆₀₀ value of 0.5, 0.3, and 0.1.

Two hundred μL of cell suspensions were mixed with 25 μL of 100 mM 4-hydroxyhippuric acid (4-HHA, Bachem 4005059, prepared in 100 mM phosphate buffer pH adjusted to 7.4) in a microtiter plate and incubated for 1 h at 37° C. After incubation, 5 μL of 100 mM 4-Aminoantipyrine (4-AAP) and 10 μL of 50 mM sodium periodate (NaIO₄) were added. The absorbance at 505 nm was recorded with a plate reader at room temperature. Final concentrations for the assay are 10 mM 4-HHA, 2 mM 4-AAP and 2 mM NaIO₄.

The samples comprising Campylobacter coli or sterile control remained colorless or slightly yellowish at all OD₆₀₀ values while in the samples comprising Campylobacter jejuni showed a red to pink color over the range of OD₆₀₀ values tested which gradual increase in intensity from an OD₆₀₀ value of 0.1 to an OD₆₀₀ value 1.1.

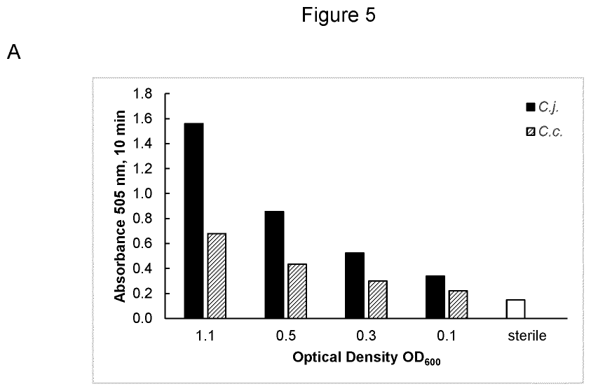

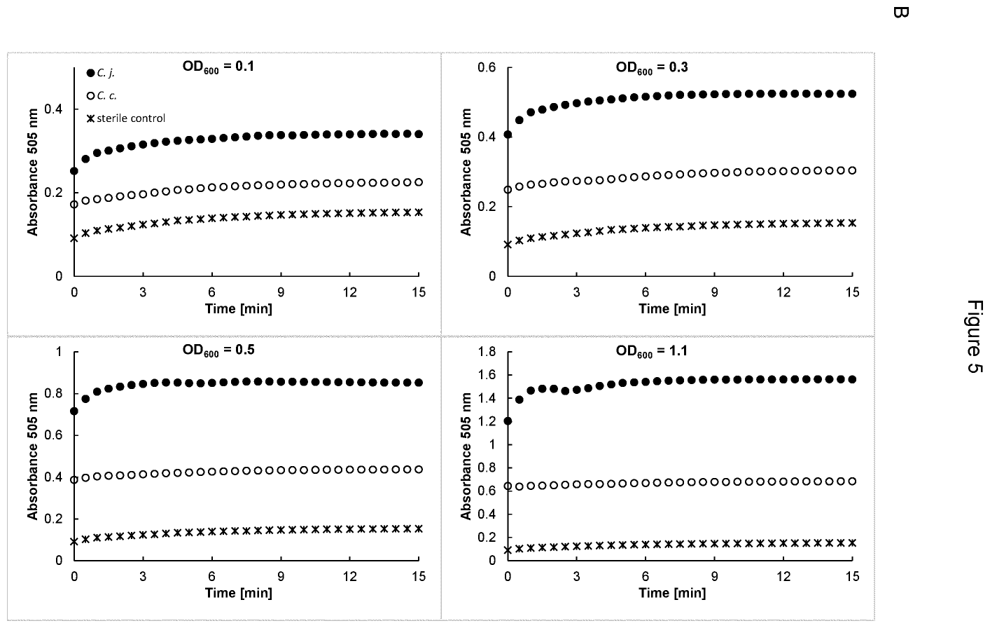

The formation of the quinoneimine compound produced by oxidative coupling of p-hydroxybenzoic acid with 4-aminoantipyrine in the presence of the hydrogen peroxide was determined by measuring the absorbance at 505 nm. The results in FIG. 5 show a higher absorbance value at all cell densities (indicated as OD₆₀₀ value) for C. jejuni compared to C. coli showing that the method is also specific and sensitive with NaIO₄ as oxidizing reagent.

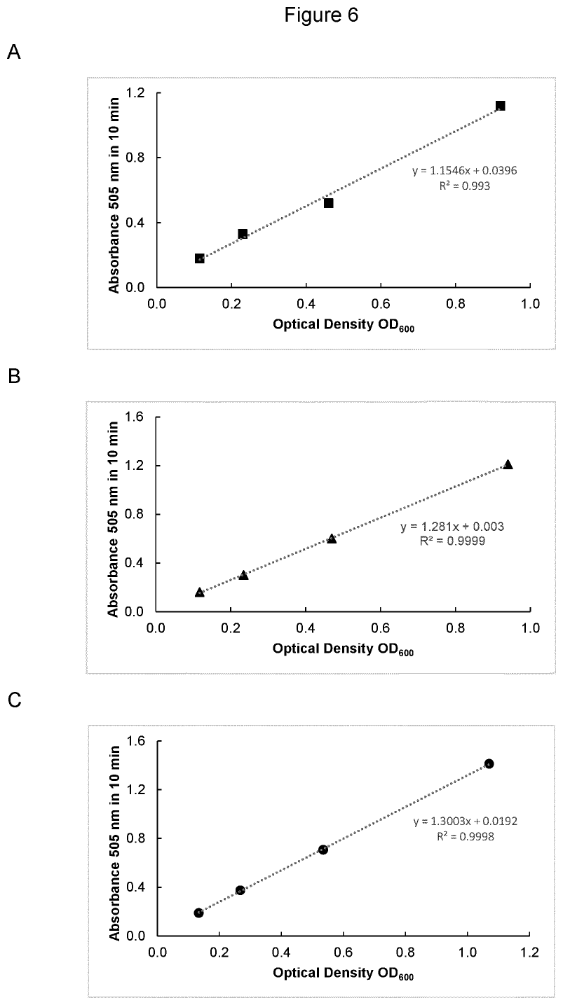

FIG. 6 shows a comparison of the linear correlation of the absorbance intensity at 505 nm and OD₆₀₀ of C. jejuni DSM4688 using different oxidizing reagents. Cell suspensions in sterile 100 mM phosphate buffer pH 7.4 were incubated with 4-hydroxyhippuric acid (final concentration 10 mM) for 1 h at 37° C., following the addition of 4-aminoantipyrine and different oxidizing reagents: A HRP/H₂O₂, B HRP/Glu/GOD, C NaIO₄. Endpoint absorbance after 10 min (absorbance of sterile control was subtracted) was evaluated.

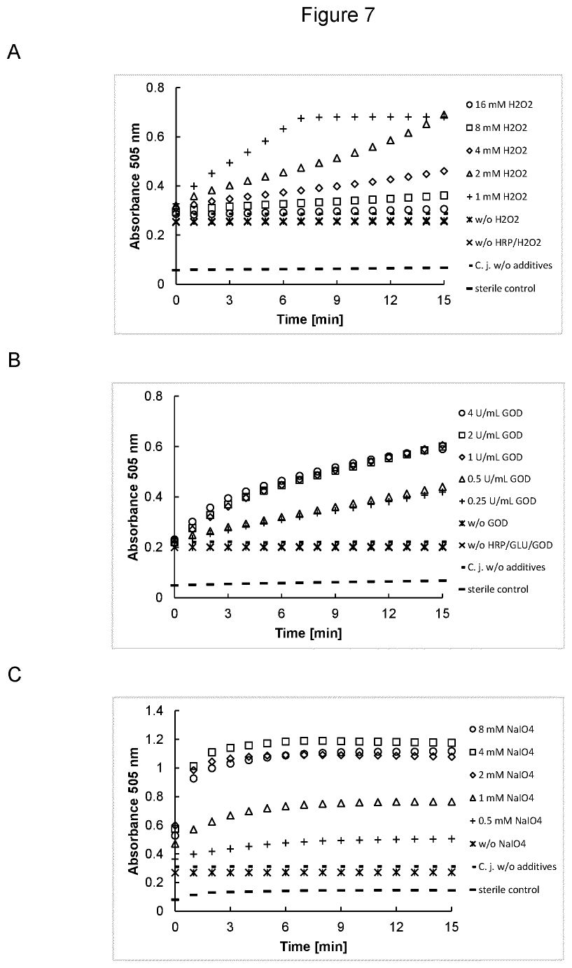

FIG. 7 shows the range of the different oxidizing reagents added by detecting C. jejuni DSM 4688 (OD=0.5 to 0.6). Cell suspensions in sterile 100 mM phosphate buffer pH 7.4 were incubated with 4-hydroxyhippuric acid (final concentration 10 mM) for 70 min at 37° C., followed by addition of A) 4-aminoantipyrine (final concentration 0.5 mM), horseradish peroxidase (final concentration 4.5 U/mL) and H₂O₂, the H₂O₂ concentration was varied from 0 to 16 mM, concentrations of 1 and 2 mM H₂O₂ showed the best performance. No coloration was observed and no signal increase was detected by 505 nm in the cell suspension without H₂O₂ or HRP/H₂O₂. B) 4-aminoantipyrine (final concentration 0.5 mM), horseradish peroxidase (final concentration 4.5 U/mL), Glucose (final concentration 10 mM) and Glucose Oxidase (GOD), enzyme activity of GOD was varied from 0 to 4 U/mL. 1 U/mL GOD is sufficient for the reaction. No coloration was observed and no signal increase was detected by 505 nm in the cell suspension without GOD or HRP/GLU/GOD. C) 4-aminoantipyrine (final concentration 2 mM) and NaIO₄, the concentration of NaIO₄ was varied from 0 to 8 mM, concentrations of 2 and 4 mM NaIO₄ showed the best performance. No coloration was observed and no signal increase was detected by 505 nm in the cell suspension without NaIO₄. Cell suspension without any additives and sterile control were included as negative controls.

### Example 6: Detection of Streptococcus agalactiae Using the Present Method

Streptococcus agalactiae ATCC®12386 and Streptococcus pyogenes ATCC®19615 were cultivated on Columbia Agar with sheep blood (Thermofischer PB5039A) at 37° C. for 48 h.

Biomass of colonies were suspended in sterile 100 mM phosphate buffer pH 7.4 and the optical density OD₆₀₀ was set to 0.5-0.6.

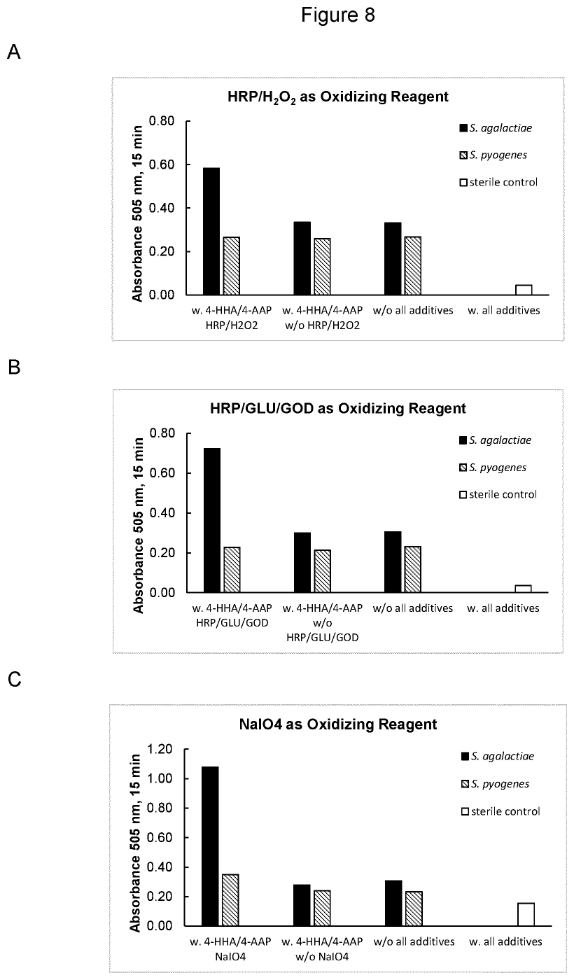

Two hundred μL of cell suspensions were mixed with 25 μL of 100 mM 4-hydroxyhippuric acid (4-HHA, Bachem 4005059) prepared in 100 mM phosphate buffer pH adjusted to 7.4) in a microtiter plate and incubated for 80 min. at 37° C. After incubation, 20 μL of the prepared mixture (12.5×) 4-aminoantipyrine (4-AAP, Merck 06800) and horseradish peroxidase (HRP, Merck 77332), 5 μL of 100 mM H₂O₂ were added. The absorbance at 505 nm was recorded with a plate reader (SpektraMax M5, Molecular Devices) at room temperature and endpoint of absorbance at 505 nm was evaluated (FIG. 8A). Final concentrations for the assay were 10 mM 4-HHA, 0.5 mM 4-AAP, 4.5 U/mL HRP and 2 mM H₂O₂.

Two hundred μL of cell suspension were mixed with 25 μL of 100 mM 4-hydroxyhippuric acid (4-HHA, Bachem 4005059, dissolved in 100 mM phosphate puffer pH 7.4) in microtiter plate and incubated for 1 h at 37° C. After incubation, 10 μL of the prepared mixture (25×) 4-aminoantipyrine (4-AAP, Merck 06800) and horseradish peroxidase (HRP, Merck 77332), 10 μL of 250 mM glucose (Merck, G8270) and 5 μL of glucose oxidase (GOD, Merck G2133) 50 U/mL were added. The absorbance at 505 nm was recorded with a plate reader and endpoint of absorbance at 505 nm was evaluated (FIG. 8B). Final concentration of 4-HHA was 10 mM, 4-AAP was 0.5 mM, HRP was 4.5 U/mL, glucose was 10 mM and GOD was 1 U/mL.

Two hundred μL of cell suspensions were mixed with 25 μL of 100 mM 4-hydroxyhippuric acid (4-HHA, Bachem 4005059, prepared in 100 mM phosphate buffer pH adjusted to 7.4) in a microtiter plate and incubated for 1 h at 37° C. After incubation, 5 μL of 100 mM 4-Aminoantipyrine (4-AAP, Merck 06800) and 10 μL of 50 mM sodium periodate (NaIO₄) were added. The absorbance at 505 nm was recorded with a plate reader at room temperature, and endpoint of absorbance at 505 nm was evaluated (FIG. 8C). Final concentrations for the assay are 10 mM 4-HHA, 2 mM 4-AAP and 2 mM NaIO₄.

The samples comprising Streptococcus pyogenes or sterile control remained colorless or slightly yellowish while in the samples comprising Streptococcus agalactiae showed a pink color. This example confirms that all the methods presented are also sensitive and specific for distinguishing hippuricase positive strain Streptococcus agalactiae from hippuricase negative strain Streptococcus pyogenes, which are important etiological factors in human illness.

1. A method of detecting a microorganism having hippuricase activity comprising:
a) providing a sample to be tested,
b) contacting the sample from step a) with p-hydroxyhippuric acid or a salt thereof, a detection reagent, preferably a reagent for phenol detection, and an oxidizing reagent, to obtain a detection mixture, wherein, if the microorganism with hippuricase activity is present, a detectable compound is produced, wherein the detectable compound is preferably produced by oxidative coupling of p-hydroxybenzoic acid with the detection reagent in the presence of the oxidizing reagent, and
c) determining in the detection mixture of step b) the presence or absence of the detectable compound, wherein the presence of the detectable compound indicates the presence of the microorganism having hippuricase activity.

2. The method of claim 1, wherein the detection reagent is a compound which modifies p-hydroxybenzoic acid to produce a chromogenic detectable compound, preferably a compound which reacts via oxidative coupling to produce a chromogenic detectable compound, wherein more preferably the detection reagent is 4-aminoantipyrine or a salt thereof.

3. The method of claim 1 or 2, wherein the oxidizing reagent comprises an oxidizing agent and a catalyst.

4. The method of claim 3, wherein the oxidizing agent is hydrogen peroxide and/or a hydrogen peroxide-generating system, wherein the hydrogen peroxide-generating system is preferably an oxidase and a substrate; and/or wherein the catalyst is a peroxidase, preferably horseradish peroxidase.

5. The method of claim 1 or 2, wherein the oxidizing reagent comprises a halogen such as a halogen salt, preferably a periodate salt, more preferably sodium periodate.

6. The method of any one of the preceding claims wherein the microorganism is from Campylobacter species, preferably selected from the group consisting of Campylobacter jejuni and Campylobacter avium; or wherein the microorganism is from Gardnerella species, preferably Gardnerella vaginalis; or wherein the microorganism is from Streptococcus species, preferably selected from Streptococcus agalactiae; or wherein the microorganism is from Staphylococcus species, preferably Staphylococcus aureus; or wherein the microorganism is from Listeria species, preferably Listeria monocytogenes.

7. The method of any one of the preceding claims, wherein step a) further comprises a culturing step, optionally wherein a pre-enrichment step precedes the culturing step, wherein the culturing step, and optionally the pre-enrichment step, preferably comprises culturing the sample under microaerobic atmosphere and wherein the microorganism is preferably of Campylobacter species.

8. The method of any one of the preceding claims, wherein the sample in step a) is selected from the group consisting of a sample from a subject, preferably from feces and/or from a body fluid, from a food, preferably from meat and/or milk, and from environment, preferably from water, dung, dust and/or industrial waste.

9. A kit for detecting a microorganism with hippuricase activity comprising:
a) a first container comprising p-hydroxyhippuric acid, a second container comprising a detection reagent, and a third container comprising an oxidizing reagent, or
b) a container comprising p-hydroxyhippuric acid, a detection reagent, and an oxidizing reagent, or
c) a first container comprising p-hydroxyhippuric acid and a second container comprising a detection reagent and an oxidizing reagent, or
d) a first container comprising p-hydroxyhippuric acid and a detection reagent and a second container comprising an oxidizing reagent, and
e) optionally instructions for contacting a sample to be tested for comprising the microorganism with p-hydroxyhippuric acid, the detection reagent, and the oxidizing reagent.

10. The kit of claim 9, wherein the detection reagent is a compound which modifies p-hydroxybenzoic acid to produce a chromogenic detectable compound, preferably a compound which reacts via oxidative coupling to produce a chromogenic detectable compound, wherein more preferably the detection reagent is 4-aminoantipyrine or a salt thereof, and/or wherein the oxidizing reagent is preferably a halogen.

11. A kit for detecting a microorganism with hippuricase activity comprising:
a) a first container comprising p-hydroxyhippuric acid, a second container comprising a detection reagent, a third container comprising a catalyst, and a fourth container comprising an oxidizing agent, or
b) a first container comprising p-hydroxyhippuric acid, a second container comprising a detection reagent and a catalyst, and a third container comprising an oxidizing agent, or
c) a first container comprising p-hydroxyhippuric acid and a second container comprising a detection reagent, a catalyst, and an oxidizing agent, or
d) a first container comprising p-hydroxyhippuric acid and a detection reagent, a second container comprising a catalyst, and a third container comprising an oxidizing agent, or
e) a first container comprising p-hydroxyhippuric acid and a detection reagent, and a second container comprising a catalyst, and an oxidizing agent, or
f) a first container comprising p-hydroxyhippuric acid, a second container comprising a detection reagent, and a third container comprising a catalyst and an oxidizing agent, or
(g) a first container comprising p-hydroxyhippuric acid, a detection reagent, and a catalyst, and a second container comprising an oxidizing agent, or
(h) a container comprising p-hydroxyhippuric acid, a detection reagent, a catalyst, and an oxidizing agent, and
i) optionally instructions for contacting a sample to be tested for comprising the microorganism with the p-hydroxyhippuric acid, the detection reagent, the catalyst and the oxidizing agent.

12. The kit of claim 11, wherein the detection reagent is a compound which modifies p-hydroxybenzoic acid to produce a chromogenic detectable compound, preferably a compound which reacts via oxidative coupling to produce a chromogenic detectable compound, wherein more preferably the detection reagent is 4-aminoantipyrine or a salt thereof.

13. The kit of claim 11 or 12, wherein the oxidizing agent is hydrogen peroxide and/or a hydrogen peroxide-generating system, wherein the hydrogen peroxide-generating system is preferably an oxidase and a substrate; and/or wherein the catalyst is a preferably a peroxidase, more preferably horseradish peroxidase.

14. Use of the kit of any one of claims 9 to 13 for detection of a microorganism having hippuricase activity, preferably hippuricase activity of an endogenous hippuricase enzyme, or for discriminating a microorganism having hippuricase activity from a microorganism without hippuricase activity.

15. Use of the kit of claim 9 or 10 in the method of any one of claims 1-2 or 5-8 or use of the kit of any one of claims 11 to 13 in the method of any one of claims 3-4 or 6-8.
